(19) United States
(12) Patent Application Publication
(10) Pub. No.: US 2003/0208113 A1
(43) Pub. Date: Nov. 6, 2003

(54) Closed loop glycemic index system

(76) Inventors: James R Mault, Evergreen, CO (US); John Sanderson, Irvine, CA (US)

Correspondence Address: GIFFORD, KRASS, GROH, SPRINKLE, ANDERSON & CITKOWSKI, PC, 280 N OLD WOODARD AVE, SUITE 400, BIRMINGHAM, MI, 48009, US

(21) Appl. No.: 10/333,383

(22) PCT Filed: Jul. 18, 2001
(86) PCT No.: PCT/US01/22720

(51) Int. Cl. A61B 5/00

(52) U.S. Cl. 600/316; 600/365; 128/921

(57) ABSTRACT

A system for assisting a person to maintain a blood glucose level between predetermined limits comprises: an electronic device, comprising a display, a clock, a memory, and a processor; and a software program executable by the processor of the electronic device, adapted to receive nutritional data of food consumed by the person, adapted to calculate the blood glucose level for the person using the nutritional data and a glycemic response model for the person, and further adapted to present the blood glucose level to the person on the display of the electronic device.

### FIELD OF THE INVENTION

This invention relates to health management, in particular, the maintenance of blood glucose (or blood sugar) levels within an acceptable range, and to the blood glucose monitoring of a person in relation to diet.

### BACKGROUND

There are significant health advantages in keeping blood glucose levels (equivalently, blood glucose concentrations) within certain limits. Excessively high blood glucose levels (hyperglycemia) have been implicated in the breakdown of capillaries (such as within the retina), and in kidney damage. The damage is possibly due to the formation of polysaccharides such as sorbitol, as discussed for example in U.S. Pat. No. 6,074,822. Low blood glucose (hypoglycemia) can lead to feelings of dizziness, nausea, extreme hunger, poor decision making, and the breakdown of diet programs. Hypoglycemia is also dangerous for people operating equipment.

Conventionally, people are somewhat unaware of their blood glucose levels, except when the levels achieve extreme values. Diabetics are required to maintain control of blood glucose levels; otherwise, serious health problems can arise. However, conventional blood glucose measurement techniques are extremely inconvenient, and hence not always used even by those for which they are medically necessary. A conventional way of measuring blood glucose is by extracting a drop of blood and reacting it with an enzyme, glucose oxidase, on a test strip. The test strip method is reasonably accurate, but only provides an instantaneous measurement of blood glucose and does not provide any indication on whether levels are rising or falling. These future trends are clearly of great importance in helping a person maintain their blood glucose levels within a healthy range. Hence, it would be extremely advantageous to provide a system by which a person can view predictions of future trends in blood glucose levels. There are non-invasive methods of blood glucose level determination, including IR absorption, analysis of interstitial fluids, and other techniques. However, even convenient non-invasive techniques, when they become available, do not on their own provide predictions of future trends.

A person's blood glucose response to food depends on the glucose available from the food, typically correlated with carbohydrate content, and on the glycemic index of the food. High glycemic index foods, such as simple sugars, cause a rapid, large increase in blood glucose levels. These foods are typically easily and quickly digested. Low glycemic index foods, such as polymerized sugars, starches, and other complex carbohydrates, cause a smaller increase in blood glucose, but the effects occur over a longer time period as the food is slowly broken down. Conventionally, the glycemic index of a food relates to the increase in blood glucose for a person after eating the food. The possible beneficial effects of modified starch based foods, with low glycemic index, are discussed by Sharp et al. in U.S. Pat. No. 5,695,903.

In U.S. Pat. No. 5,913,310, Brown describes a video game system which may be used as part of a blood glucose control system. However, this system does not include the recording of foods eaten or otherwise consumed.

Integ Inc. of Roseville, Minn., has described methods of extracting interstitial fluid from the dermis, so as to analyze blood glucose levels, for example as described in U.S. Pat. Nos. 6,203,504, 6,152,889, 6,120,464, 6,080,116, 5,879,367, 5,879,310, 5,823,973, 5,820,570, 5,746,217, 5,682,233, and 5,582,184. Glucose levels within the interstitial fluid were shown to correlate well with glucose levels within the blood. A device described in U.S. Pat. No. 6,080,116 to Erickson et al. comprises a housing having at least one microneedle sized so as to penetrate the epidermis into the dermis, but not to penetrate beyond the dermis into the subcutaneous layer. An array of such microneedles may be used to extract quantities of interstitial fluid from the skin.

Recently, Cygnus developed a wrist mounted blood glucose measuring device called the GlucoWatch. Glucose is drawn out of interstitial fluid using reverse iontophoresis. Neutral glucose molecules are extracted by the flow of positive ions out of the skin, from beneath the dead cell layer of the epidermis. Glucose concentration is then determined by an enzymatic reaction with glucose oxidase.

A diabetic care overview wristwatch has been described by Olsen in U.S. Pat. No. 6,188,648. This device alerts the person to eat certain numbers of carbohydrate at certain times, and has other alerts for taking medicine and measuring blood glucose. It does not perform diet logging, and does not account for the glycemic response of the person to foods. This device is useful for setting a rigorous schedule of actions, but is not adapted to respond to a person's variable schedule.

Other conventional diabetes management systems and methods are described in the following patents (U.S. Pat. Nos. unless otherwise indicated): 4,731,726 to Allen, 5,019,974 to Beckers, 5,695,949 to Galen et al., 5,822,715 and WO00/18293 to Worthington et al., 5,971,922 to Arita et al., 5,997,475 to Bortz, and 6,027,692 to Galen et al.

Conventional blood glucose measurement techniques (including analysis of other body fluids providing related data), which may be advantageously used in embodiments of the present invention, are described in (U.S. Pat. Nos. unless otherwise indicated) 5,077,476 to Rosenthal, 5,139,023 to Stanley, 5,640,954 to Pfeiffer et al., 5,666,956 to Buchert, 5,871,695 to Khartchenko et al., 5,954,685 to Tierney, 5,989,409 to Kurnik et al., 6,023,629 to Tamada, 6,080,116 to Erickson et al., 6,091,976 to Pfeiffer et al., 6,120,464 to Racchini et al., 6,144,869 to Berner et al., 6,152,889 to Sopp et al., 6,166,807 to Kawamura et al., and WO99/46600 to Ullman et al. Glycemic index, in relation to food compositions, is discussed in 5,234,906 to Young et al., and 5,695,803 to Sharp. Fluorescence-based glucose sensors are disclosed by Ullman et al. in WO99/46600.

The contents of U.S. Patents and published Patent Applications mentioned herein, are incorporated herein by reference.

The contents of the following U.S. Provisional Applications are incorporated herein by reference: 60/219,070 (filed Jul. 18, 2000), 60/219,512 (filed Jul. 20, 2000), 60/225,454 (filed Aug. 15, 2000) 60/228,680 (filed Aug. 29, 2000), 60/240,185 (filed Oct. 13, 2000), 60/243,621 (filed Oct. 26, 2000), 60/257,138 (filed Dec. 20, 2000), and 60/269,063 (filed Feb. 15, 2001).

### SUMMARY OF THE INVENTION

Embodiments of the present invention provide methods of blood glucose management systems that allow a person to view projected future levels of blood glucose, so as to assist the person to keep blood glucose levels within a predetermined range, such as a medically advisable healthy range. Systems are provided by which a person can view future projections of blood glucose levels in based on actions or planned actions, receive advance warnings of possible excursions of blood glucose from the predetermined range, and receive advice on diet, exercise, behavior modification, or medical treatments that can help the person prevent the blood glucose level excursions into unhealthy ranges.

In one embodiment, the system comprises a portable computing device such as a personal digital assistant (PDA), having a software program (such as a calorie management program or other health maintenance program) having a glycemic modeling capability, such as a glycemic response algorithm. The term PDA will be used for convenience to refer to a portable computing device. The software program can be adapted to receive blood glucose measurements of the person. Blood glucose levels can be determined by any convenient technique for comparison with the glycemic response predictions of the software program. The software program can be advantageously modified from the program described in U.S. Provisional Application 60/240,185 to James R. Mault and others, so as to have a glycemic response algorithm, to record blood glucose levels, and to access a component response database by which the blood glucose response of a person to a meal can be determined by identifying meal components and determining the glycemic response of the person to the components. Diet logging methods are described in more detail later.

The person records meal information using the diet log capabilities of the software program. The software program receives food data, and uses a glycemic response model to calculate the change in blood glucose level for the person over time, projecting future values and presenting the future values to the person on a display. The glycemic response model can be individualized by monitoring the blood glucose response of the person to standard meals, for example using a conventional glucose tolerance test, or a glucose tolerance test adapted to determine the response to other meals of different nutritional balance. The projected blood glucose levels, as determined by the software program, can be compared with measurements of blood glucose performed by the person at intervals. This allows the accuracy of the model to be checked, and can be used to modify the model used so as to provide more accurate blood glucose projections. Comparison of modeled and actual blood glucose can also identify errors and omissions in creating a diet log. The glycemic response model can be modified in a closed loop method, whereby a combination of diet logging, blood glucose modeling, and blood glucose measurement is carried out, with measured blood glucose levels and model-based predictions compared, allowing the model used to be modified so as to improve accuracy.

The person may consume one or more prepackaged (or otherwise nutritionally well defined) meals during the day. These may include prepackaged low calorie or low fat meals, barcode labeled items, meals for which the vendor supplies accurate nutritional information, and other well identified meals. For example, as part of a health management program the person may receive prepackaged breakfast and lunch meals, and it would then be relatively simple for the person to enter the time and meal identity into the software program.

For better control of blood glucose, a person may be supplied by prepackaged meals supplied by a health management business. These meals may be labeled with conventional nutritional data, including for example carbohydrate, fat, protein, fiber, sugar, and complex carbohydrate content, along with glycemic response parameters. These parameters may be conventional glycemic indexes, or improved parameters as described in more detail later so as to allow the calculation of the person's glycemic response to recorded meals. The parameters may be scaled using an individual correction factor determined from for example glucose tolerance tests.

Diet logging can be performed at the time a meal is consumed, however for people not suffering from diabetes it may be acceptable to enter food items after the event. The person enters food data, which may comprise food identity, food components, time of consumption, or planned time of consumption if the meal is planned but has not been consumed. If behavior is identified which is leading to unacceptable excursions in blood glucose levels, the person may be advised on changes, for example by a computer expert system. Meals may also be entered for planning purposes, to determine whether planned meals and exercise programs may blood glucose excursions from a predetermined range.

The following example illustrates how an embodiment of the present invention can be used by a person suffering from Type 1 diabetes, which arises when insulin secreting cells of the pancreas are destroyed by an autoimmune response. After a meal, blood glucose levels rise, but without the presence of insulin body cells are not able to extract glucose from the blood, and therefore become energy starved. The body may attempt to obtain energy by metabolizing fatty acids from fat tissues, leading to a condition called ketoacidosis, in which the blood and urine contain high levels of ketone bodies, and ketones such as acetone are present in the breath. Such a person conventionally uses insulin injections to control blood glucose levels, but the level of control may be crude. The damage to eyes, nerves, etc. caused by excessive deviations of blood glucose levels may be reduced if the blood glucose level excursions from a predetermined range are prevented.

The person carries a portable computing device, such as a PDA, which receives blood glucose levels from a blood glucose sensor. The blood glucose sensor may be a device clipped on to the earlobe of the person, using at least one semiconductor laser or light emitting diode to emit wavelengths in the near-IR. These wavelengths are transmitted through or reflected by blood-containing tissue, and then detected by a near-IR detector. A number of emissive devices may be used, or electrical modulation of a single device may be used to obtain multiple wavelength emission. For example, two wavelengths may be chosen, one absorbed and one not absorbed by blood glucose, the absorption ratio then being used to compute the blood glucose level of the person. Other blood glucose sensors are known in the art, and improved sensor systems are discussed below. The blood glucose data can be transmitted from the sensor to the PDA using the Bluetooth wireless protocol.

The person enters meals consumed (in terms of food content and time of meal), and planned meals, into the PDA using the diet log functionality of a software program running on the PDA. The software program further uses a glycemic response model for the person to predict future blood glucose levels, based on the planned meals and possible insulin injections. The quantity and time of injections can be optimized for glucose level stability. The software program may provide interactive feedback to modify a future meal, exercise, or administration of medication so as to prevent excursions of blood glucose from a predetermined range. The person may execute insulin injections manually, or an insulin pump or other insulin delivery system may be used. An insulin pump can be in communication with the PDA, for example using Bluetooth radio communication, so that the PDA receives data on time and quantity of injections, and possibly can command an insulin injection of a specific quantity at a certain time.

The following example illustrates how the system may be used by a person with Type 2 diabetes. In this condition, body tissues become less sensitive to insulin action. If the pancreatic cells are unable to increase insulin production sufficiently to overcome this drop in sensitivity, then the symptoms of type 2 diabetes occur. Conventionally, treatment may include diet modifications, oral drugs, and (less commonly) insulin injections. The person enters meals consumed, and planned meals, into the PDA. Blood glucose levels are projected using a glycemic response model. If oral drugs (or any other treatments) are being used, the model of future blood glucose levels is used to optimize the effect of the treatment, for example through modifying dose quantity and/or treatment time. The person can be prompted to confirm that planned meals are consumed. The person can also be prompted to take medications at calculated optimum times. If blood glucose levels appear to be heading for higher levels than predicted by the model, additional medication or physical activity may be suggested by the software program. The software program running on the PDA can be used to control an insulin pump, for example in the manner described in the example above.

Weight loss has important health benefits for persons suffering from Type 2 diabetes. However, it can be difficult for the person to plan an effective exercise program, as there is significant danger that blood glucose levels may fall to overly low levels (hypoglycemia). However, the system described here facilitates the planning of a safe exercise program. The glycemic response model for the person is used to predict blood glucose levels over a period of time. If exercise is planned, the planned exercise can be entered into the PDA, which can suggest times for the exercise based on blood glucose levels. The effect of physical activity on glycemic response can be determined, enabling the PDA to suggest limits on the planned exercise, additional snacks (for example fruit juice, candy) which it might be advisable to consume, or any other adjustments so as to avoid the onset of hypoglycemia.

In the case of the person using insulin injections to control blood glucose levels, the PDA can be used to used to control injections, either through information displayed to the person, or by radio communication with a subcutaneous insulin pump, as in Example 1.

The following example illustrates the use of respiratory analysis in glycemic response monitoring systems. The Gas Exchange Monitor (GEM) invented by James R. Mault, described in U.S. Pat. App. U.S. patent application Ser. No. 09/630,398, incorporated herein by reference, is an indirect calorimeter which allows determination of metabolic rate. In one embodiment of the GEM, oxygen partial pressure in the respired gas flow is measured using a fluorescence quenching sensor. The fluorescence of an irradiated polymer film is quenched by interactions with oxygen. Ultrasonic transducers are used to determine flow rate, and the integration of flow volumes and oxygen partial pressure, corrected to standard conditions, allows oxygen volumes in respired gases to be determined. The metabolic rate, in combination with estimates or measurements of physical activity, allows determination of the energy requirements of a person. The PDA may use this information in suggesting meals as part of a glycemic control diet. A fluorescence of a fluorophore-doped polymer film may be quenched by other respiration components, such as nitric oxide, ketones/aldehydes, and glucose molecules in respired gases, allowing other respiration components to be determined simultaneously with metabolic rate determination.

A ketone sensor allows molecules such as acetone in exhaled gas to be detected. This indicates fat metabolism, for example due to heavy exercise. The presence of breath acetone combined with high blood glucose levels is indicative of ketoacidosis, characteristic of Type 1 diabetes. Hence, a sensitive breath ketone sensor may be used to warn the person about the onset of this condition.

The concentration of glucose molecules in exhaled breath is in equilibrium with the glucose levels of blood in the lungs. The initial component of exhaled air may be discarded from the analysis to ensure only deep alveolar exhalation is monitored. The respiratory glucose sensor may complement or replace blood glucose sensing. The respiratory analyzer can be in communication with the PDA, for example using a wireless protocol such as Bluetooth (Ericsson AB).

An improved software program, comprising glycemic control and calorie management functionalities forms part of an improved weight control system. Calorie management functionality can comprise diet logging, diet control (such as menu suggestions), activity logging, and activity control (such as exercise program generation). The metabolic rate of the person is measured using, for example, an indirect calorimeter such as the GEM. A physical activity program is devised, taking account of the health of the person. Combined with the general activity levels in the person's life and the person's metabolic rate, this allows determination of the number of calories required by the person for weight maintenance. A weight loss program requires less calorie intake than that expended by the person in physical activity and metabolism.

The software program running on the PDA can then be used in planning a weight control program for the person. The person may request certain meals, and the software program may suggest others to maintain a certain caloric intake combined with other nutritional requirements. The software program can also comprise an individualized glycemic response model for the person, allowing estimation of future blood glucose levels based on planned and consumed meals. Meal content and time changes can be suggested to limit blood glucose excursions.

A diet can break down at times of very low blood glucose level. An embodiment of the present invention described here reduces this possibility by reducing the occurrence of such times through diet planning. For example, from a person's glycemic response, and planned meals, it might be calculated that the blood glucose level will hit a low point at a certain time in the afternoon. The PDA can then prompt the person to take a small snack at this time for example fruit. Snacking can be part of an improved diet program if it is used sensibly to help regulate blood glucose levels.

In one embodiment, the system used by the person comprises a PDA and an activity monitor in communication with the PDA, for example using the Bluetooth wireless protocol. The PDA has a software program with diet logging and glycemic response prediction functionalities. The person enters meals consumed, or planned meals, into the PDA and receives feedback, possibly including: meal modification (for planned meals); future meal modification; exercise; long term meal planning issues; optimum times for medication, exercise, etc; and other matters. The measured glycemic response can also be used to determine the calorie content of the food consumed, as an alternative to manual logging of calories consumed.

Hence, a method for blood glucose control in a person, comprises: determining a glycemic response for the person relative to consumption of food; using the glycemic response parameters to determine a glycemic response model for the person; using the glycemic response model to project blood glucose levels for the person; and providing feedback to the person based on the projected blood glucose levels.

A method for determining the glycemic response of a person, comprises: recording food consumed by the person over a period of time; recording time-dependent blood glucose levels for the person over the extended period of time; and using the time-dependent blood glucose levels to determine the glycemic response of the person to different foods. The time dependency of the blood glucose can be modeled, for example using multi-parameter fits.

A weight control method for a person, comprises: determining a metabolic rate of the person; determining an activity level of the person; determining a glycemic response of the person; recording foods consumed by the person on a portable electronic device; and providing feedback to the person using the portable electronic device whereby the person uses the feedback received to modify their diet and exercise program in order to consume a projected number of calories, while eating a healthy diet and avoiding extreme blood glucose deviations from an acceptable level.

A system for calorie management and blood glucose control for a person comprises: a portable electronic device; an activity level sensor, in communication with the portable electronic device; and a software program running on the portable electronic device, the software program having diet logging functionality and blood glucose prediction functionality.

A method for controlling the blood glucose level of a person comprises: determining a glycemic response for the person to at least one meal; providing a software program, running on a computing device, having a food recording function, and further having a glycemic response algorithm from which the person's blood glucose response to recorded food consumption can be predicted; and providing feedback to the person based on predicted blood glucose level

A system for controlling a blood glucose level of a person comprises: a blood glucose sensor, providing data correlated with the blood glucose level of the person; a computing device, having a display; a software program, running on the computing device, adapted to receive data from the blood glucose sensor, further adapted to receive food consumption data from the person, and further having an algorithm so as to display a blood glucose projection on the display.

A method by which a person can control their blood glucose comprises: entering food consumption data into a software program, the software program running on a computing device, the software program adapted to record food consumed and to calculate a glycemic response; viewing the calculated glycemic response on a display of the computing device; and performing an action in response to the calculated glycemic response, so as to maintain blood glucose within predetermined levels. The action may comprise the administration of insulin, the administration of glucagon; the performing of exercise, eating, or contacting a medical professional.

A method of predicting a blood glucose response of a person to a food comprises: recording a food using a food identity and a food quantity; determining food components of the food using a database, the database correlating the food identity with the food components; and predicting the blood glucose response of the person to the food using an algorithm, the algorithm correlating the blood glucose response of the person with the food components and with the food quantity. The algorithm may further correlate the blood glucose response of the person with a physical activity level of the person. The algorithm may further correlate the blood glucose response of the person with the metabolic rate of the person.

A weight control method for a person comprises the steps of: determining a metabolic rate of the person; monitoring dietary consumption of the person; monitoring an activity level of the person; determining a caloric balance for the person; determining a hunger index for the person; and using the hunger index and caloric balance to suggest food intake; whereby the person is assisted in their weight control program.

A method of assigning a hunger index to a person comprises the steps of: determining a blood glucose level for the person; determining a satiety level for the person; determining a psychological level for a need to eat for the person; and combining the determined levels using an empirical relationship whereby a hunger index is assigned to the person, and used in a weight control program.

A system for assisting a person to maintain a blood glucose level between predetermined limits comprises an electronic device, for example a portable computing device such as a PDA, comprising a display, a clock, a memory, and a processor; and a software program executable by the processor of the electronic device, adapted to receive nutritional data of food consumed by the person, to calculate the blood glucose level for the person using the nutritional data and a glycemic response model for the person, and further adapted to present the blood glucose level to the person on the display of the electronic device. The program can receive diet log data from the person, for example food identities, then correlate the food identities with nutritional data using a database. The software program can be further adapted to receive time data related to the time of consumption of food (including beverages, nutraceuticals, and other consumables) by the person, and to receive data relating to planned consumption of food. Blood glucose levels can be calculated for a plurality of times, and presented as a table or graph. An activity monitor, in communication with the electronic device, can providing a signal correlated with a physical activity level of the person. An insulin delivery system, adapted to deliver insulin into the person by injection, infusion, aerosol, nasally, orally, using an implant, or any other method, can be in communication with the electronic device, so that the software program can initiate operation of the insulin delivery system, for example in response to projected blood glucose levels. A glucose sensor, in communication with the electronic device, can provide a signal correlated with the blood glucose level of the person. The glucose sensor can comprise at least one microneedle adapted to draw interstitial fluid from the person to a fluorescence sensor, so that the fluorescence sensor provides a fluorescence signal correlated with glucose concentration in the interstitial fluid; an analog-to-digital converter, providing a digital representation of the fluorescence signal; and a transmitter, adapted to transmit the digital representation of the fluorescence signal to an electronic device. The electronic device can comprises a wireless receiver, and the blood glucose sensor can comprise a wireless transmitter which transmits the digital representation of the fluorescence signal to the wireless receiver of the electronic device. The glucose sensor can comprises a fiber optically coupled to a fluorescence sensor in contact with lymph fluid, with interstitial fluid, or blood.

A method for assisting a person to maintain a blood glucose level within a predetermined range comprises: receiving food identity data, corresponding to food consumed by the person, for example using a diet log; correlating food identity data with nutritional data, for example using a database; calculating the blood glucose level for the person for at least one future time using the nutritional data and a glycemic response model; and presenting the calculated blood glucose level to the person. Time data relating to food consumed, or planned to be consumed, by the person, can also be received. The glycemic response model for the person can include parameters provided by a glucose tolerance test, and the calculation of the blood glucose level for the person can further use glycemic index data correlated with the food identity data. Alerts can be provided if the calculated blood glucose level falls outside a predetermined range, and advice can be provided to the person to assist the avoidance of this condition.

A method of determining a glycemic response model for a person comprises: receiving diet log data, wherein the diet log data comprises the nutritional data of foods consumed by the person; measuring blood glucose levels of the person at intervals; determining an initial glycemic response model for the person, using the diet log data and the measured blood glucose levels; determining a calculated blood glucose level using the glycemic response model and diet log data; providing a comparison of the calculated blood glucose level and a measured blood glucose level; modifying the glycemic response model based on the comparison, so as to improve the accuracy of the glycemic response model; and repeating the above steps, until the glycemic response model provides an acceptably accurate prediction of blood glucose levels using diet log data. After the model is believed to be accurate, it may be tested at intervals, for example using a blood glucose measurement every day, few days, or longer interval. The diet log data can be correlated with glycemic parameters of foods using a database.

A method of determining a glycemic response model for a person comprises: estimating an initial glycemic response model for the person; recording foods consumed by the person; recording blood glucose levels for the person at intervals; modifying the initial glycemic response model so as to provide a modified glycemic response model of improved accuracy, wherein accuracy is determined from a comparison of calculated blood glucose levels provided by the model with measurements of blood glucose levels. The initial glycemic response model can be determined using demographic factors associated with the person, conventional databases of glycemic indexes of foods, or other information.

### BRIEF DESCRIPTION OF THE DRAWINGS

FIG. 1 is a schematic diagram of a glycemic response system.

FIG. 2 illustrates a specific embodiment of a glycemic response system.

FIG. 3 illustrates the response of blood glucose level to a meal.

FIG. 4 illustrates a menu-type meal entry on a portable electronic device.

FIG. 5 illustrates blood glucose feedback on a portable electronic device.

FIG. 6 illustrates a system for monitoring gestational diabetes.

FIG. 7A shows a system for monitoring blood glucose.

FIG. 7B shows a schematic of a system for monitoring blood glucose.

FIG. 8 shows a wrist-mounted device in communication with a PDA.

FIG. 9 shows a gestational monitoring system.

FIG. 10 shows a subject monitoring system.

FIG. 11 shows a schematic of a combined diet log and glycemic modeling system.

FIG. 12 illustrates a glucose tolerance test.

FIG. 13 shows a display on a portable computing device, by which a person can select foods to create a diet log.

FIG. 14 shows projected blood glucose levels presented to a person.

FIG. 15 shows projected blood glucose levels presented to a person.

FIG. 16 shows an alert screen presented to a person.

FIG. 17 shows a graph of hunger index versus time.

FIGS. 18A, 18B, and 18C show blood glucose response models.

FIG. 19 illustrates a software program for calculating calorie balance and blood glucose projections.

### DETAILED DESCRIPTION

Embodiments of this invention provide a person with improved control over their blood glucose levels. This improved control is important in diabetes management, and can also be combined with a weight control program by providing a software program combining glycemic response modeling with diet logging functionalities. A glycemic response model for a person, which is used to predict changes in blood glucose level based on the recording of foods consumed, or foods planned to be consumed, can be modified for improved accuracy by comparison with actual measurements of blood glucose. Further, the glycemic response model for a person can be determined by relating blood glucose measurements with diet log records, in terms of food consumed and time of meals, in a closed loop system by which the model can be constantly improved based on new comparisons of projected and actual blood glucose levels.

System Embodiments

We describe a system to assist the control of the blood glucose level of a person. In one embodiment, the person's blood glucose levels are monitored, while nutrition intake is recorded in a diet log. The person's glycemic response is then determined, and used in an improved program to control blood glucose. This invention describes how diet and possibly medication and exercise can be included in such a program.

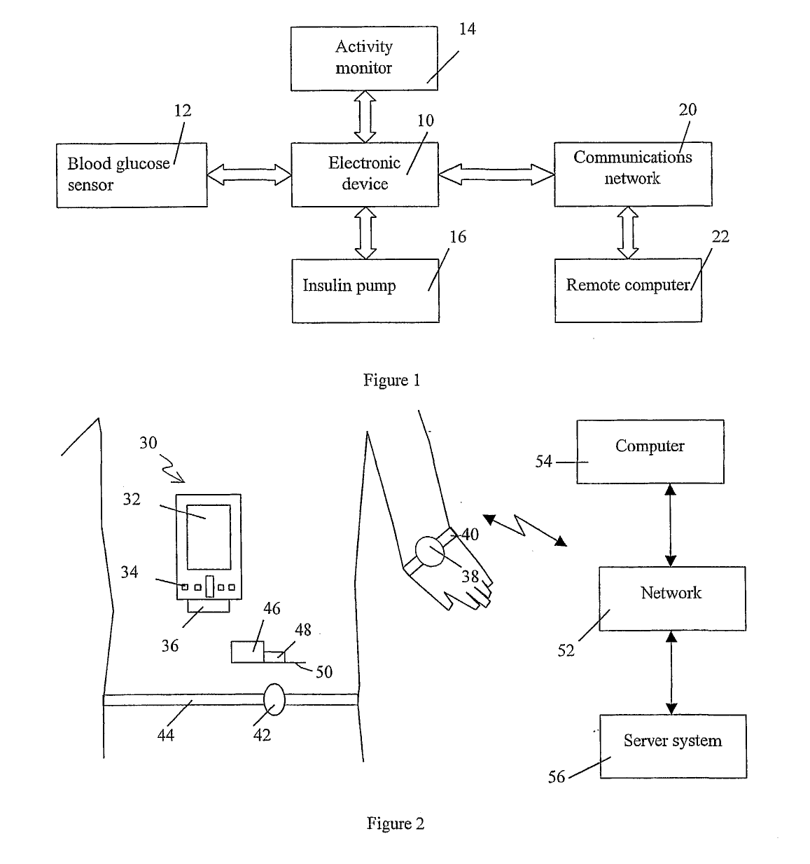

FIG. 1 shows a schematic of a system embodiment comprising an electronic device 10, a blood glucose sensor 12, a physical activity monitor 14, an insulin pump 16, a communications network 20, and a remote computer system 22.

Electronic device 10 can be carried by the person, for example a portable computer, personal digital assistant (PDA), wireless phone with computational functionality, visor-mounted device, or other device. The electronic device 10 can be a PDA such as a Palm Pilot. The electronic device can be a portable electronic device or system carried by the person, or a device or system combining multiple functionalities, such as: a personal digital assistant (PDA); an electronic book (e-book); a portable computer; any portable Internet access device; a wireless phone; a wristwatch, adapted to record data, for example to monitor physiological properties, or act as a diet log (it may also act as a wireless internet access device, or be otherwise adapted); a headset, providing for example visual and audio communication to the person, and recording audio and/or visual information; a body mounted activity monitor (for example pedometer) adapted to record data; any portable electronic device with data logging capabilities, for example an electronic organizer; or some combination of the above or other devices.

The portable electronic device receives data, and provides feedback to the person. For convenience, the device will be referred to as a PDA, though it may contain a number of functionalities in addition to those of a conventional PDA, such as the Palm V (Palm Computing). The portable device can be in communication with a communications network, for example through a wireless Internet connection or similar. The PDA may be a single device, or may be a system of separate components, for example (for non-limiting illustrative purposes only) a collar-mounted microphone transmitting to a device carried in the pocket, which itself transmits images to a projection unit mounted on a visor, and audio to a hidden earpiece. The system configuration of the PDA is not critical to this invention.

The glucose sensor 12 may be a monitoring kit as used in a doctor's office or the person's home, conventionally involving the drawing of blood and an analysis of blood glucose levels. Data from such tests may then be manually entered into the PDA. However, repeated use of such kits is inconvenient.

Sensor 12 can be carried by the person and provides blood glucose measurements at intervals. For example, sensor 12 can uses ion flow to draw glucose molecules out of the blood into a gel disk containing the enzyme glucose oxidase, generating hydrogen peroxide, which is detected and a numerical value displayed. This technique is used by GlucoWatch developed by Cygnus (Redwood City, Calif.). The sensor can then transmit the numerical value of blood glucose to the PDA 10, for example using a wireless method. Alternatively, sensor 12 may use spectroscopic detection of a glucose oxidase reaction. Sensor 12 may also use in-vivo spectroscopy techniques to measure blood glucose, for example as described in U.S. Pat. Nos. 6,067,463 (Jeng), 6,049,727 (Crothal), 5,660,163 (Schulman), and others. For example, IR absorption or reflection properties of skin or tissues may be determined, for example using a finger, toe, wrist, skin membrane, earlobe, nose, cheek, retina, or other body part. A blood analysis sensor may be built in to a module which plugs in to the PDA. For example, using a non-invasive spectroscopic technique, the PDA/module combination may be placed against part of the body to obtain a blood glucose reading. The sensor may also be placed under the skin, with wireless communication, such as using the Bluetooth protocol, to the PDA. The sensor may be powered by internal batteries, by light using a photoelectric effect, by temperature gradients using a thermoelectric effect, by radiation (e.g. from AM radio stations, electric mains, or radiated by the PDA or other portable device), and may be either continuously powered or only powered at times a reading is required. The sensor may record data to a memory card which plugs into the PDA.

In one embodiment, the glucose sensor transmits the blood glucose level data to the PDA using a wireless communication protocol such as Bluetooth (Ericsson Components AB). Communication methods between sensor 12 and PDA 10 might include Bluetooth, IR transmission, IEEE 802.11(b), wireless Ethernet, optical methods, acoustic methods, or other wireless methods, or using a cable connection, electrical interface, or manual entry of data into the PDA using any input mechanism, such as keys, a stylus, voice recognition, and the like. The PDA may request a blood glucose reading from the sensor.

Physical activity monitor 14 may comprise a body mounted accelerometer, and can transmit data to the PDA correlated with the physical activity level of the person. Monitor 14 may used to determine the effect of physical activity on glycemic response. For example, after consuming a meal of known nutrition content, the person may engage in a repetitive physical activity and the effect of this on the blood glucose response can be determined. The physical activity monitor may be in communication with the PDA 10 using a variety of methods, including Bluetooth wireless transmission, an IR link, cables, other electromagnetic or electrical methods, transfer of memory cards, manual entry of data, ultrasonic methods, and the like.

The PDA 10 is in communication with the insulin pump 16, using a wireless communications protocol such as Bluetooth, cable, or through human intervention. The PDA receives data on the time and quantity of insulin injections. The PDA may transmit a request to the pump for an insulin injection, possibly after confirmation by the person. The insulin pump can be omitted if not required.

Remote computer system 22 may be a remote server, physicians computer, home computer, or other computer system accessible over the communications network 20. The PDA can access the communications network, such as the Internet, using a wireless connection. Data can be transferred to the remote system over the communications network 20, allowing examination by a dietician, physician, other specialist, or by the person themselves using the PDA or any computer. Feedback, generated by a specialist or computer expert system, may be transmitted to the PDA from the remote computer over the communications network. In an emergency situation, the PDA may send out a request for medical assistance. A global positioning system (GPS) may also be added to the PDA, to facilitate medical rescue in an emergency.

The PDA 10 may further communicate with a home computer, interactive television, or other entertainment device using for example a wireless Internet connection, IR link, Bluetooth, memory card transfer, or other electrical/electromagnetic methods.

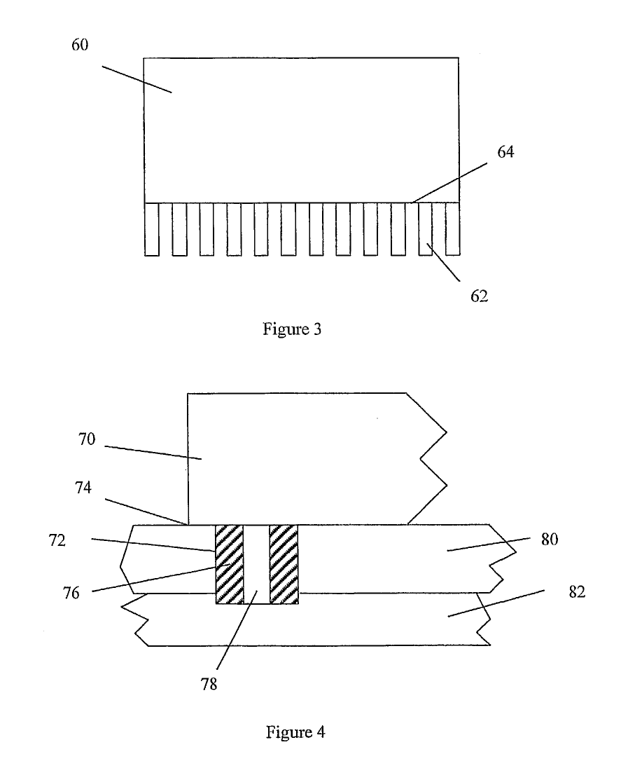

FIG. 2 shows a system embodiment, comprising portable computing device 30 having a display 32, data entry mechanism 34, and wireless transceiver 36; a wrist-mounted glucose sensor 38 having a strap 40; activity monitor 42 supported by the person's body using belt 44; insulin storage and dispensing device 46 comprising a subcutaneous needle 50 and delivery system 48 such as a pump. The portable computing device 30 is in linked to a communications network 52, allowing communication a remote computer system 54, which may be located at the person's home, doctor's office, or other remote location. A remote server system 56 is also accessible through the communications network 52, and can be used to store a database of physiological parameters, activity, and diet relating to the person, for medical, nutritional, or computer expert system analysis. The wrist mounted sensor 40 and activity monitor 42 can transmit data to the portable computing device 30 through a wireless method. The device 30 can be carried in a pocket of the person's clothes, or on a belt clip, or other convenient method.

Functionalities of system elements can be combined. For example a wrist mounted device can combine the functionality of a glucose sensor and a portable computer. An earring-like device could combine the functionality of a glucose sensor and a device to give audible feedback to the person, perhaps as an extension of the PDA, and in for example Bluetooth wireless communication with it. The PDA may have a plug in module which analyzes blood. Other combinations are possible.

Glucose Sensor

FIG. 3 shows a blood glucose sensor housing 60 having a plurality of microneedles 62 projecting from a surface 64 of the housing 60. The microneedles 62 are hollow, and are of a length such that they penetrate through the outer layer of the skin of the person (through the epidermis), so that they are in contact with interstitial fluid below the outer layer of the epidermis. To reduce discomfort, the microneedles can be a length which does not penetrate the dermis.

A person holds the housing 60 to their skin, so that the surface 64 lies closely against the skin, such that a plurality of microneedles 62 puncture the skin surface. Interstitial fluid is drawn up through the microneedles 62 into the housing 60. A variety of techniques can be used to extract interstitial fluid from the skin, including iontophoresis, other electrical methods, ultrasonic methods, capillary action, thermal irradiation, and suction. The housing may contain a plunger, be deformable, or provide another mechanism for generating negative pressure within the microneedles so as to draw interstitial fluid out of the skin. The housing may also be strapped to the body of a person, or otherwise held against the skin.

FIG. 4 shows a surface 74 of a blood glucose sensor housing 70 resting against the surface of the skin of a person. For convenience, a single microneedle 72 is shown in cross section, having a wall 76 and a hollow center 78. The microneedle 72 is shown penetrating the epidermis 80 of the person, so as to locate the end of the microneedle below the epidermis 80. Skin layer 82 contains interstitial fluid, which may be analyzed. Interstitial fluid is then drawn up the microneedle 72 into the housing for analysis. To avoid disturbing nerve endings and blood vessels of the person, the needle should be short enough so as not to penetrate the dermis.

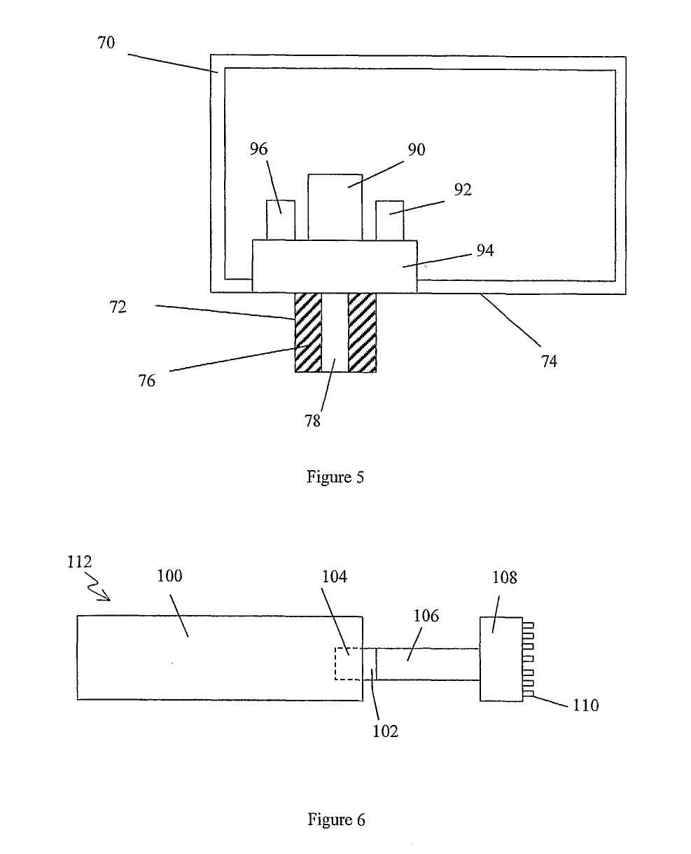

FIG. 5 shows a microneedle 72 with a hollow center 78 in fluid communication with fluorescent matrix 94. Matrix 94, for example a fluorophore doped polymer or sol-gel, is excited by light emitting diode 90. Photodetector 96 detects fluorescent radiation from a sensing region of the matrix, which is affected by the presence of analyte in the interstitial fluid drawn from the skin. A reference photodiode 92 is used to provide a reference signal from an reference region of fluorescent matrix, into which the analyte does not enter. For example, a semi-permeable membrane can be used to prevent glucose molecules from entering reference regions of the matrix. A number of microneedles such as 72 can pass fluid to a fluorescent matrix. The sensor housing contains at least one fluorescent matrix. In other embodiments, a plurality of separate analyte and reference matrix regions can be used.

The blood glucose sensor housing can also contains a sensor control circuit, for example one advantageously adapted from the oxygen sensor control circuit disclosed in U.S. patent application Ser. No. 09/630,398, a processor, a memory, and a wireless communication module. The wireless communication module can be a Bluetooth protocol transceiver, adapted to transmit blood analyte levels to other devices, such as electronic devices, insulin pumps, portable computers, diet software, other medical equipment, and the like.

Excitation radiation, for example from a light emitting diode, may be passed along the hollow center of a microneedle. A fluorescent matrix may be supported by the end of the microneedle, or line the wall surface of a microneedle, so as to be in contact with interstitial fluid, allowing fluorescent detection of analytes in the fluid.

In other embodiments, one or more optical fibers are used instead of or in addition to microneedles. The fibers have a length sufficient to penetrate the epidermis. A fluorescence sensing chemistry region is incorporated into the end of the fiber, so as to respond to the analyte of interest, such as glucose. For example, the end of the fiber may be a fluorophore-doped polymer permeable to the analyte of interest. Excitation radiation is passed down the fiber, so as to excite the fluorescent end of the fiber, and fluorescence is returned along the fiber to a detector. Optical filters can be used in addition to detectors.

FIG. 6 shows a microneedle module adapted to act as an accessory to a PDA. A microneedle array 110 is formed on one surface of the microneedle module 108, which is connected to an analysis module 106, the analysis module being connected to an interface 102 which plugs into a slot 104 within the housing 100 of PDA 112. At intervals, possibly alerted by the PDA, the person places the microneedle array against the skin allowing interstitial fluid to flow into the module. Analysis is performed within the analysis module, using any appropriate method. In one embodiment, the interstitial fluid is drawn into a gel layer, where it is analyzed for blood glucose using the conventional glucose oxidase reaction, or another analytical technique. Infrared and Raman spectroscopy can also be used for noninvasive analysis of blood glucose levels. A module may be placed against the skin to detect the IR signature of glucose of fluid beneath the skin, or a spectroscopic mechanism can be included into the analysis module described above so as to detect levels of glucose within the interstitial fluid extracted from the skin.

Analytical techniques which may be used to measure glucose concentrations in blood, interstitial fluid, urine, or other body fluids and tissues include: techniques based on the glucose oxidase reaction; other enzymatic reactions; IR (particularly near-IR), Raman, and optical-UV spectroscopy; fluorescence; colorimetry; attenuated total reflection, for example from a surface adapted to bind glucose; micromechanical methods such as detecting changes in resonance frequencies on analyte absorption, or having surfaces adapted to bind glucose with other detection methods; optical rotation and ORD; refractometry; fiber sensing; laser absorption; enzymatic methods such as using the glucose oxidase reaction; biochemical effect detection; or other appropriate techniques. Fluid samples may be removed from the body, or analyzed within the body.

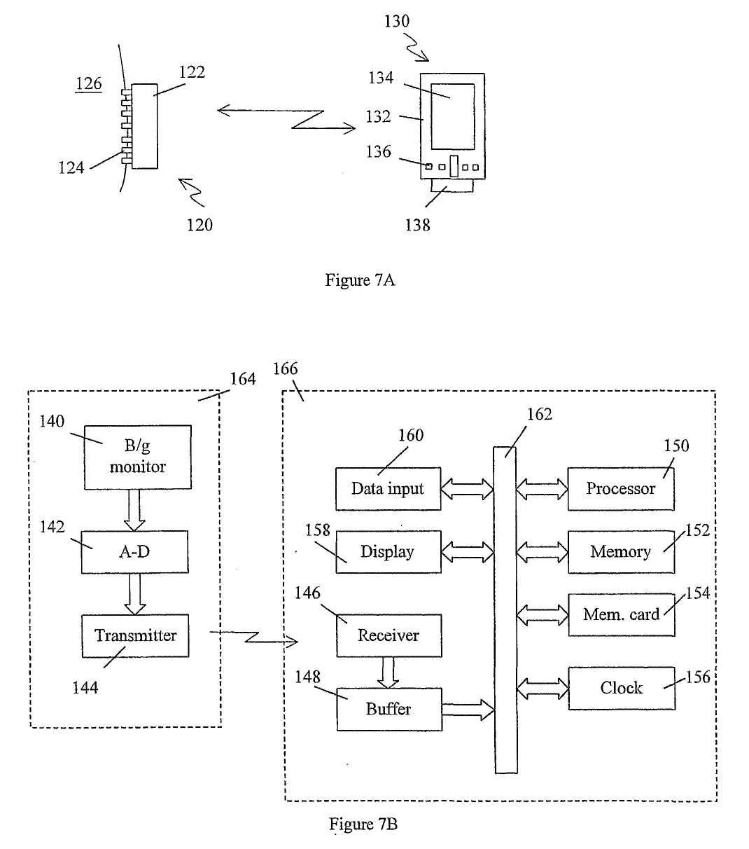

FIG. 7A shows a blood glucose analysis module having a microneedle array placed against the skin of a person and in wireless communication with a PDA. The analysis module, shown generally at 120, has a housing 122 having a microneedle array 124 on one surface. The array is placed against the skin 126 of a person. The PDA 130 has housing 132, display 134, data entry mechanism 136, and an accessory communication module 138 used to communicate with the analysis module 120. Communication may use a cable link, or a wireless communication such as the Bluetooth protocol developed by Ericsson and other companies. The communications module may be contained within the housing of the PDA. The analysis module 120 may be powered by radiation generated by the PDA or other electronic device, for example by inductive coupling, capacitive coupling, thermoelectric, photoelectric, or piezoelectric effects. In other embodiments, a direct electrical interface may be formed between the PDA and the microneedle analysis module 120. Suction, heat, pressure, radiation, chemicals, or other external factors may be advantageously applied to the skin to enhance interstitial fluid production.

The module 120 contains a wireless transceiver for transmitting data to another device, such as the PDA 130. The measurements of blood glucose are received by the PDA, and provided to the software program used for diet logging and glycemic response prediction. The measurements can be used to update and refine a glycemic response model.

FIG. 7B shows a schematic of a possible embodiment of a system blood glucose monitoring system, an analysis module 164 comprising a blood glucose sensor 140 providing an electrical glucose signal correlated with blood glucose levels, an analog-to-digital converter 142 providing a digital representation of the glucose signal, and a wireless transmitter 144 transmitting the digital representation of the glucose signal to other devices in communication with the analysis module. A portable computing device 166 which can communicate with the analysis module comprises a wireless receiver 146, a data buffer 148, a processor 150, a memory 152, a memory module (e.g. memory card) interface 154, a clock 156, a display 158, and a bus 162.

In one embodiment, interstitial fluid is drawn out of the dermis of the skin into an analysis means. Fluid can be drawn out of the skin through one or more inlet microneedles, and returned to the dermis through one or more outlet microneedles, so as to provide a flow of interstitial fluid through the module. The fluid can flow through an array of microspheres, such as silica or polymer microspheres, the surfaces of which have a molecular species which fluoresces in the presence of glucose. Alternatively, the fluid may flow through a capillary or microcapillary having an inside surface lined with a fluorescence film sensitive to the concentration of glucose in the fluid. The fluorescence is detected as a measure of glucose concentration, using a detector and excitation radiation source (which can be located within the housing) possibly in comparison with a reference region not exposed to interstitial fluid flow. IR radiation may also be waveguided along capillaries, or scattered by microspheres, so as to spectroscopically detect glucose. In other embodiments, the analysis module uses the detection methods developed by Cygnus for the GlucoWatch, as discussed above. The module may alternatively draw blood or lymph fluid into the housing for analysis, for example using a micromechanical pump. The analysis module may alternatively perform non-invasive analysis of blood glucose, for example by clipping to an ear lobe or other skin flap and transmitting near-IR radiation through the skin flap. An analysis module with a fluorescence sensing region may be optically accessed by an external device having an excitation radiation source and a fluorescence detector, for example a fluorescence sensor can be contained within a transparent housing and embedded in the skin.

Blood glucose levels are correlated with glucose levels in the interstitial fluid, and also with levels in the lymph system. A microneedle system can be used to draw fluid from lymph vessels or from a lymph nodes, for analysis using techniques described here, or other techniques known in the art. An optical fiber having a fluorescence sensing end can be inserted into the skin so that the sensing end is located within interstitial fluid, a lymph vessel, or a lymph node. A sensor can be implanted into the skin, lymph vessel, or lymph node so as to provide a signal correlated with blood glucose, for example using transponder methods which are known in the art. Blood glucose sensors can also be combined with immunological sensors for health monitoring, and with other physiological monitoring methods such as discussed later.

Glucose can also be detected in the breath of a person, for example using a respiratory analyzer such as an indirect calorimeter provided with a fluorescence glucose sensor, and the data correlated with blood glucose

Wrist Mounted Device

James R. Mault and others have described wrist mounted devices for diet and exercise logging, for example as described in U.S. Provisional Application Nos. 60/207,051 and 60/243,621. A wrist mounted diet log and/or activity log system is usefully combined with a glucose meter for use in a glycemic control system.

For close control of blood glucose, a person may choose to be supplied with prepackaged meals or other nutritionally well quantified meals. For example, breakfast and lunch can be supplied to the person as prepackaged meals. At breakfast and lunchtime, the person can scan the food package with a barcode scanner in the wrist mounted device, or in communication with it. In other embodiments, a portable electronic device such as a PDA can be adapted to read data from a package or barcode using different methods, such as bar-code scanning, optical character recognition, and the like. Diet logging methods are discussed in more detail elsewhere in this application. Combined with real time blood glucose measurements determined using a blood glucose sensor within the wrist-mounted device, future trends in blood glucose can be calculated using a glycemic response model. An alarm can sound if it is predicted that levels will be exceeded. The wrist-mounted device has a display, which can be used to show time, and present other data to the person. This display, or an additional display, can be used to show codes, icons, graphics, and alphanumeric data to the person to indicate actual or projected excursions from a healthy range of blood glucose. An alarm may sound if levels are predicted to be exceeded in the near future.

The wrist-mounted device may have one or more of the following functionalities: glucose measurement; barcode scanner; a memory containing nutritional data, correlated with food identity data such as names, numeric codes, UPC barcode data, and the like; voice recognition capability for data input; a display, such as an electronic display or a laser projection display for large scale information projection against a surface; an IR port, for communication with other devices; a wireless transceiver such as Bluetooth enabled transceiver; a camera, for recording food images; a connection to a communications network, such as a wireless Internet connection, for sending and receiving data which may include nutritional information associated with non-recognized UPC codes or other data; a memory module port for transfer of data; an electrical connector, for communicating with another electronic device; a processor for running a software program having diet log, activity log, and glycemic response model functionalities; a wireless phone functionality; activity recorder, for example using pulse, or activity time flagging. Other functionalities, such as the alert systems described by Olsen in U.S. Pat. No. 6,188,648, herein incorporated by reference, can also be included.

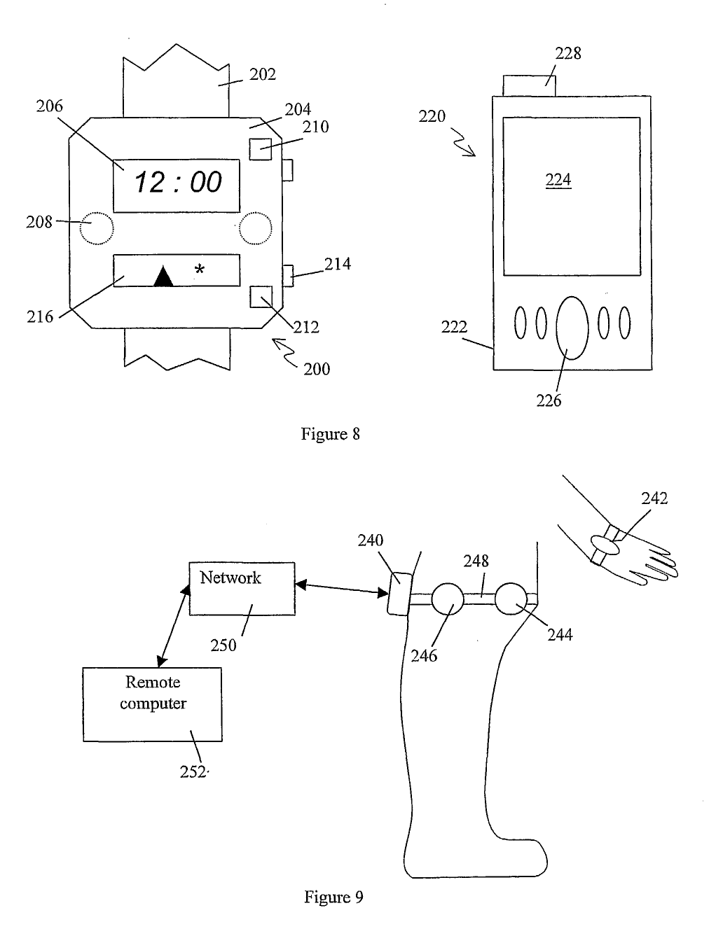

FIG. 8 shows a wrist-mounted device 200 having a strap 202; housing 204; display 206; electrodes on the rear surface 208 for interstitial fluid extraction, with corresponding fluid analysis functionality within the housing; a barcode scanner 210; an IR port 212, for communication with a PDA or other device; and a graphics display 216. Buttons such as 214 are provided for setting the time, mode changes, and the like. The IR port allows communication with PDA 220. The PDA 220 has housing 222, a display 224, data entry mechanism 226, and an IR port 228. Other wireless communication methods, such as the Bluetooth protocol, or a cable link, can be used between the wrist mounted device and the PDA.

The wrist-mounted device is adapted to monitor the blood glucose level of the wearer, for example using the methods and apparatus as described in U.S. Pat. Nos. 6,144,869 to Berner et al., 6,023,629 to Tamada, 5,989,409 to Kurnik et al., and 5,954,685 to Tierney, the contents of which are incorporated by reference. The wrist-mounted device is provided with a mechanism for drawing interstitial fluid out of the dermis into a medium, such as a gel or porous matrix, within the main housing, and a means for performing a test for blood glucose level using the fluid within the medium, for example the glucose oxidase reaction described in U.S. Pat. No. 5,108,819, herein incorporated by reference. Interstitial fluid can be withdrawn from the dermis using pressure, suction, capillary action, ion flow, peristalsis, osmotic flow, dialysis, heat treatment, abrasion, or other appropriate method. Analysis of the fluid for glucose concentration may use any convenient technique known in the art or described elsewhere in this specification.

The display capability of the PDA is used in diet logging, diet planning, and glycemic response modeling, and a software program running on the PDA can be adapted to use data received from the wrist-mounted device. Also, data can be entered into the PDA and transmitted to the wrist-mounted device for use in glycemic response prediction. A PDA may communicate in a similar manner with other devices adapted to monitor blood glucose levels or otherwise be carried by the person, such as earrings, finger mounted devices, and other skin mounted monitors.

Gestational Blood Glucose Control

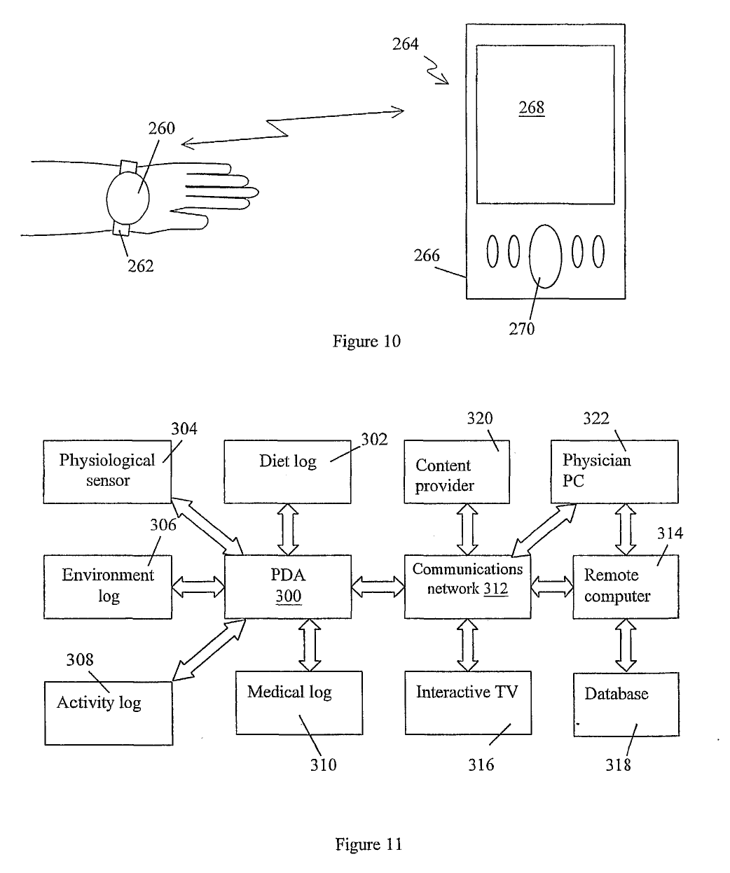

FIG. 9 shows a glycemic control system integrated with a fetal monitoring system for pregnant women. Electronic device 240 is shown mounted on a belt 248 but can be any portable electronic device such as a PDA. The electronic device 240 receives data from a blood glucose sensor 242 which is illustrated as a wrist mounted device but can be any convenient location, such as mounted on belt 248. The belt 248 can be placed around the waist of the woman. Belt 248 also carries a fetus monitoring sensor system 244, which can comprise for example fetal activity monitors, fetal heart sensors, ultrasound imaging sensors, a contraction sensor, other sensors, or a combination of sensors. An activity monitor 246 is also mounted on belt 248, and can be an accelerometer, pedometer, pulse rate meter or other device providing a signal correlated with activity. The activity monitor can be integrated into the wrist mounted device, for example as a pulse rate sensor. Other physiologic sensors can be added to the system. Communication between the electronic device 240 and the sensor/monitor devices 242, 244, 246 can be achieved using the Bluetooth wireless protocol. Communication between the PDA and the remote computer system 252 can be using a wireless connection to a communications network 250, such as the Internet, so that the data collected by the PDA can be transmitted for example to a web site accessible by a physician. In other embodiments, all portable system elements are carried on a belt, and the belt provides communication between system elements for example using integrated cables. A battery can also be carried by or built into the belt.

The electronic device 240 is used as a diet log and to store blood glucose readings. The device provides dietary feedback to the mother based on a measured and modeled (or estimated) glycemic response. The device also collects data from physiological sensors monitoring the health of the baby and/or mother. An audible alert can sound if projected or actual blood glucose levels deviate from a predetermined range.

Subject Monitoring

Embodiments of the present invention allow a caregiver to monitor and control the blood glucose level of a subject, such as a patient, child, invalid, intubated patient, and the like. The caregiver records foods consumed and corresponding times using the software program. The software program can have a glycemic response algorithm for the subject.

FIG. 10 shows a blood glucose monitoring system 260, in the form of a watch, supported on a monitored subject using a strap 262, providing data to a PDA 264 in possession of the caregiver. The PDA 264 has housing 266, display 268, and data entry means in the form of a keypad 270. Other data entry mechanisms can be used, such as a stylus, touch pad, tracker device, biofeedback system, roller-jog dial, voice recognition, and the like. Wireless data transmission can be used, such as Bluetooth (Ericsson) or IR transmission. Electrical connections may be used at intervals, or data read off a display of the wrist-mounted device. The PDA has a software program, such as a calorie management program, having a glycemic response algorithm for the subject. The software program can be adapted for diet logging and glycemic response modeling for a plurality of subjects. Monitoring device 260 can further be any wearable, portable, or implanted analysis device. The caregiver records meals consumed by the subject, estimates activity levels (or receives data from a physical activity monitor worn by the subject), and is alerted to possible or actual blood glucose excursions from a safe range. The caregiver can control diet, exercise, insulin administration, or other subject treatment or condition so as to maintain a healthy status of the subject.

System Including an Interactive Television

FIG. 11 shows a general schematic of a system embodiment of the present invention. PDA 300 is carried by the person. The PDA 300 contains, or receives data from, a diet log 302 (for example software on the PDA, a voice recorder, a notepad, a digital camera, or other methods as described elsewhere in this specification), a physiological sensor 304 (for example a pulse rate sensor, or other physiological sensor as described elsewhere in this specification), an environmental log 306 (for example, ambient temperature measurement or as described elsewhere in this specification), a physical activity monitor 308, and a medical log 310. Various monitoring, recording, or sensing systems can be used in creating the various logs. The system can include the diet log and activity monitor; the other sensors or loggers can be omitted. Data collected is transmitted via a communications network 312 to a computer system 314. Feedback to the person may be provided by the PDA 300, an interactive television 316, or other electronic device. Data collected from the PDA (and possibly from other sources) is stored in a database 318. Data provided by the PDA 300 or database 318 may be used to determine the content viewed on the interactive television 316. The content is provided by a content provider 40, for example a broadcaster, cable service, or server. A physician may view collected data via the physician's computer 42. A software program having diet log, activity log, glycemic response model, or hunger index model (to be described later) may reside on the PDA 300 or on the computer system 314 so as to be accessed using the communications network, for example using a PDA, computer, web appliance, interactive TV, or other device which can communicate with the communications network.

The PDA may interact with other electronic devices, for example to enhance the quality of feedback to the person. For example, an interactive television or web-TV may be used to provide customized audio-visual feedback to the person, based on the collected lifestyle data (where lifestyle data refers to some or all of diet data, activity data, physiological monitoring data, medical data, etc.). Customized streaming video may be provided by a remote computer system, and viewed by the person using an Internet content viewing device.

The following system enhancements are also possible. The PDA can also be used for health and/or alertness monitoring, for example using at least one physiological sensor. If serious problems arise, emergency services may be called automatically.

The PDA may be used to order goods and services related to the weight control program, for example as suggested by a computer expert system.

The PDA may communicate with a remote computer system, for example for sending data to a database or web-site, for examination by authorized persons or systems. For example, a physician may monitor the condition of a patient remotely.

Diet Log Software

Software may be supplied to existing owners of PDAs, or a PDA having software may be supplied by the health management business. Other portable computing devices with data entry capabilities and some display capability may also be used, such as a suitably adapted wireless phone, electronic book, pager, and the like. In this example, the term PDA is used to refer to a portable computing device, having a display or other data presentation mechanism, data entry procedures, memory, and a processor.

In use, the person records food consumed using a software program running on a PDA, having a diet log function, for example as described in U.S. Provisional App. 60/240,185 (filed Oct. 13, 2000). Data entry can be made using any convenient method, such as stylus entry, keyboard entry, menu selection methods, entering product codes, barcode scanning, imaging, optical character recognition, entering user defined codes representing common meals, making voice records, voice recognition software, using a roller jog entry (as used on Sony devices), or using any convenient method. Meal data can be entered as food consumed, food about to be consumed, or food planned to be consumed in the future. At intervals, the person can enter a blood glucose level determined using any convenient technique. These techniques will be described in more detail later. The software program, working from previously entered blood glucose levels, and any previous diet log information, calculates a projected curve of blood glucose levels from the food items recorded. This curve is shown on the display of the PDA, or using the display capabilities of any device in communication with the PDA. If the projected curve goes outside a predetermined healthy range, actions can be recommended to the person, these include changing planned meals, not eating the whole meal, replacing high glycemic index foods with low glycemic index foods such as complex carbohydrates, administering an insulin injection (which may be done automatically by a device in communication with the PDA), eating a snack at a later time (the time can be suggested by the software program, and the PDA may be used to alert the person to eat the snack), performing exercise (an optimum time, duration, nature, and intensity of exercise may be recommended), cancellation of exercise, future meal suggestions, performing medical emergency procedures, infusion of medications, dialysis with blood glucose or other blood component removal, or other actions which are performed so as to maintain blood glucose levels within a healthy range. The organizer capability of the PDA can be used to plan meals, activities, administration of medication, appointments, and other actions. The PDA can synchronize data with a personal computer, so that the enhanced display and data entry capabilities of the PC can be used to enter and review displayed information.

Glycemic Response Test

An important aspect of this invention is the measurement of a person's blood glucose response to diet, and use of the response data, in the form of an individualized model, to help maintain blood glucose levels within a predetermined range.

A health management business may provide diet log software so as to run on a computer belonging to the person. Alternatively, the business may provide a computer, for example a portable computing device such as a PDA, on which the software program is installed. The computer can have a display, a microprocessor, memory (in the form of RAM, ROM, and a data storage device), and data entry mechanisms such as a keyboard, buttons, stylus, voice entry, roller-jog dial, track ball, finger tracking device, and the like.

The software program allows the person to create an accurate record of foods consumed. The software program has diet logging capabilities, for example as described in U.S. Provisional Application 60/240,185 to Mault et al. The software program further includes a glycemic modeling algorithm which predicts the effects of consumed food items on the consumer's blood glucose level. The software program can be adapted to receive measurements of the person's blood glucose levels at intervals. These levels may be determined by conventional pinprick measurements, interstitial fluid analysis, lymph analysis, urine analysis, retinal response, other non-invasive techniques as they become available, or other blood component analysis methods.

In one method, the person is subjected to at least a conventional glucose tolerance test so as to provide data for the glycemic response algorithm. The person fasts for a period (such as overnight), has their blood glucose level measured, consumes a meal, for example a known quantity of carbohydrate such as a measure of fruit juice, and then the person's blood glucose response to the food intake is determined from one or more blood glucose level measurements corresponding to times after the consumption of the carbohydrate. For example, tests may be performed on blood drawn one, two, and four hours after consumption of the carbohydrate. However, other food tolerance tests can be devised, for example using foods of different nutritional balances such as foods containing significant quantities of fiber, fat, protein, etc. Non-invasive blood glucose measurement can be performed at more frequent time intervals after food consumption. For example, a health management business may provide a person with a non-invasive meter for the duration of the glycemic response measurements. This meter may be more expensive than a meter used routinely by the person, but may only be required for a week or so.

Demographic information (such as age, weight, height, gender, ethnic background and the like) and other information (such as metabolic rate, medication programs, dietary additive use, stimulant use, fitness level, cardio-pulmonary performance, hydration level, and other physiological data) can be used to predict glycemic response, or to take the results of limited glucose tolerance tests and apply them to a wider range of conditions. Demographic data can also be used to make an initial guess at the person's glycemic response, which is then refined by comparing the model response and predictions to actual measurements of instantaneous, medium term, or long term blood glucose levels using any appropriate method.

A person might eat a meal of known nutrition content, possibly after a period of fasting. This may be a standard glucose tolerance test, in which the person consumes a drink containing 75 grams of glucose. Blood glucose levels are measured after the meal is consumed, and the body glycemic response is determined to this food is determined. Meals of other nutrition content (for example different fat, fiber, carbohydrate, etc. levels) may also be consumed at later times, allowing the glycemic response of the person to be determined for meals as a function of nutrition content. For example, dietary fiber does not directly contribute to blood glucose, but can modify the rate of carbohydrate absorption. This effect can be quantitatively measured and used in a glycemic response model. The glycemic response may also be determined for complete meals typically consumed by the person, for example a sandwich from the local deli for lunch. The glucose response data is stored by the PDA, where it is used for predictive purposes in a diet and blood glucose control system.

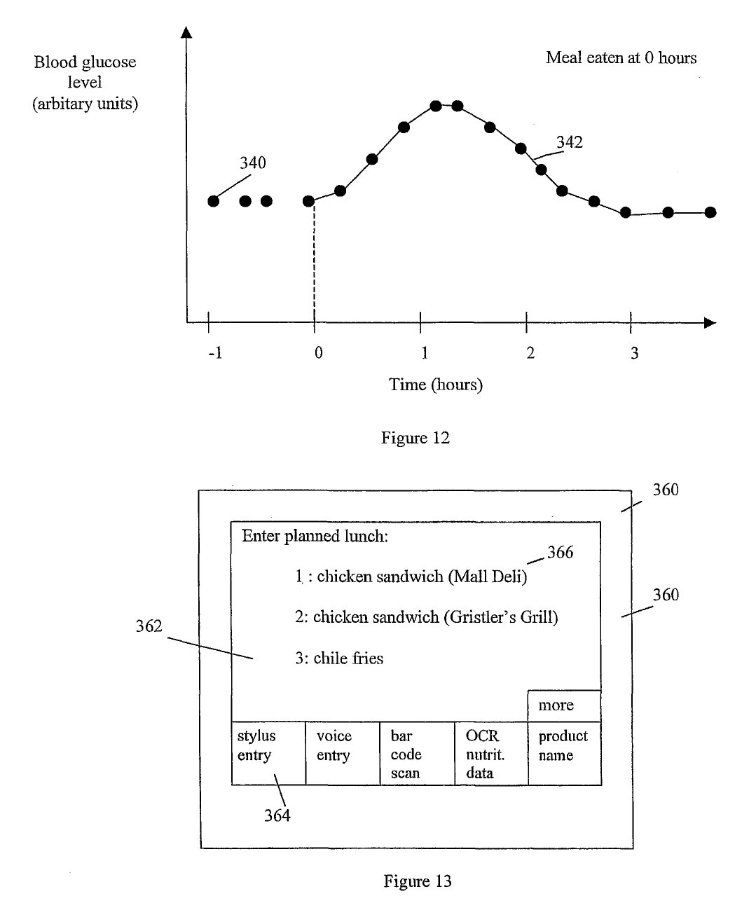

FIG. 12 shows a typical response of blood glucose to a meal, taken at a time labeled zero. Points 340 represent blood glucose level measurements. Curve 342 is fitted to the data by a computer (either a PDA, or other electronic device), and used in a model of the person's glycemic response. The model can be stored on a portable electronic device used, and can be used to help a person plan diet, medication, exercise, and with other physiological/lifestyle decisions. This enables a method for blood glucose control in a person, as glycemic response parameters can be determined for the person relative to consumption of a representative variety of foods. Glycemic response parameters encompass the time-dependent blood glucose response of the person in terms of (for example maximum response), time of maximum response relative to the meal, rise slope behavior, decay slope behavior, decay time (for example, to a baseline level) and any other parameter required to give an adequate description of the blood glucose response over time. A glycemic response model is then developed on the basis of a set of glycemic response parameters, relating to a representative selection of foods. The model is then used to project blood glucose levels for the person, based on food consumed and recorded, or planned meals.

The person may take orally-administered insulin control drugs, for example sulfonylureas (to stimulate extra insulin formation), metformin (to reduce glucose formation by the liver and increase blood glucose uptake), acarbose (to inhibit carbohydrate digestion), or derivatives of the now-withdrawn Rezulin (to decrease cell resistance to insulin). In this case, the glycemic response of the person is measured including the effect of such drugs, and medication effects can be included into a glycemic response model.

The person's glycemic response is hence measured under different conditions and incorporated into a model for that person. Parameters which may be included in the model may include: nutrition content (for example fat, protein, fiber, complex carbohydrates, simple sugars, etc.); physical activity; response to drugs; response to insulin; time of day; temperature (body, ambient); other environmental effects; person's weight (if changing).

The PDA may also be used to keep a diet log for the person over an extended period of time, for example one month, during which blood glucose readings are made at appropriate intervals, for example every half-hour, perhaps more frequently just after a meal. The glycemic response of the person can then be calculated from the blood glucose readings and the recorded diet log using multi-parameter fits. The response to different meals will overlap in the blood glucose response over time, but using data fitting techniques, the responses to different meals may be resolved, particularly if the same meal is consumed on several different days. The response to a meal may then be broken down into the response to individual components, particularly if the same component occurs in different meals. The fitting of the overall glycemic response data to modeled responses to individual meals, and then possibly to responses due to meal components, is an example of a multi-parameter fit to the glycemic response data.

Alternatively, or ill addition, average blood glucose measurements may be made using for example urine analysis (which may give data related to the previous few hours blood glucose levels); or glycohemoglobin or fructosamine tests. In conjunction with the diet logs, such average blood glucose tests give an indication of what dietary trends have caused problems, and this information can be used to give advice in the future as to what foods to avoid.

Use of Glycemic Model in Glycemic Control

Once the glycemic response has been modeled, the model can be used in an improved system to control blood glucose levels. A person may eat a meal, enter the nutrition data into the PDA, receive projections of future blood glucose levels, and advice on how to avoid dangerously high or low levels. The person may also enter future planned meal, or meals, into the PDA, and receive advice concerning modifications to the meal content, the meal times, or treatment (for example medicine) steps to avoid problems. The PDA can provide feedback on how to minimize blood glucose excursions outside of a safe range, for example modified meals (for example adding fiber to a meal to slow blood glucose rises), optimum medication times, optimum insulin injection times and quantities, small snacks at times of low blood glucose, etc.).

FIG. 13 illustrates a possible display 362 on an electronic device 360 (such as a PDA) during entry of a planned meal into the device. The display comprises a menu display 366 and data entry method options 364. A menu-type entry scheme can be convenient, particularly if the range of foods consumed is not that great. Menus can be divided into breakfast, lunch, dinner, and snack sub-menus, and other sub-menus. Data entry methods can include keystroke entry, voice entry (using voice recognition software), stylus entry, optical character recognition applied to packaging/nutrition information, barcode scanning (from the package, menu, etc.), wireless transponder methods (for example from transponder chips mounted on packaging), and the like. A PDA would have a software program allowing diet logging running on it, or can be used to interact with a software program over a communications network.

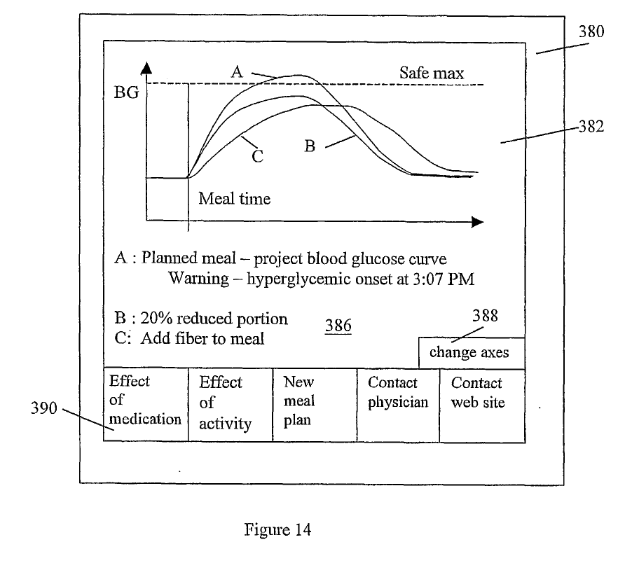

FIG. 14 illustrates possible feedback to the person from the display 382 of an electronic device 380, such as a PDA. The modeled glycemic response of a person allows blood glucose levels to be projected at times beyond the consumption of a meal, or number of meals, using for example a software program having blood glucose prediction functionality. The graph 386 shows three projected blood glucose curves, labeled A (corresponding to a planned meal), B (corresponding to a reduced portion size), and C (corresponding to addition of fiber to the meal). Curve A leads to an excursion of blood glucose above a predetermined safe maximum level. Curves B and C can be generated by the computer so as to present health alternative meal plans which avoid blood glucose level excursions. Option selectors 390 allow the person to investigate the effects of medication (such as insulin), activity (such as exercise), and new meal plans (such as modified recipes) on the effect of projected blood glucose. The person can also contact a medical professional (for example, if serious problems are projected), or receive advice from a web site accessible over a communications network. Option selector 388 allows the person to view the graph on a different scale, otherwise modify the graphical presentation, obtain tabular data, or plot long term blood glucose averages.

If blood glucose levels are expected to exceed upper limits, feedback is given on how this might be avoided, for example meal modifications (for example less simple sugar content, higher fiber content, other nutritional modifications, smaller portions, different eating times, etc.), insulin injections, medication, exercise, etc. If projected blood glucose levels fall below a lower acceptable limit, actions suggested might include a snack (with optimum time calculated), glucagon injections, meal modifications (for example more slowly digested food), changed meal times, etc.

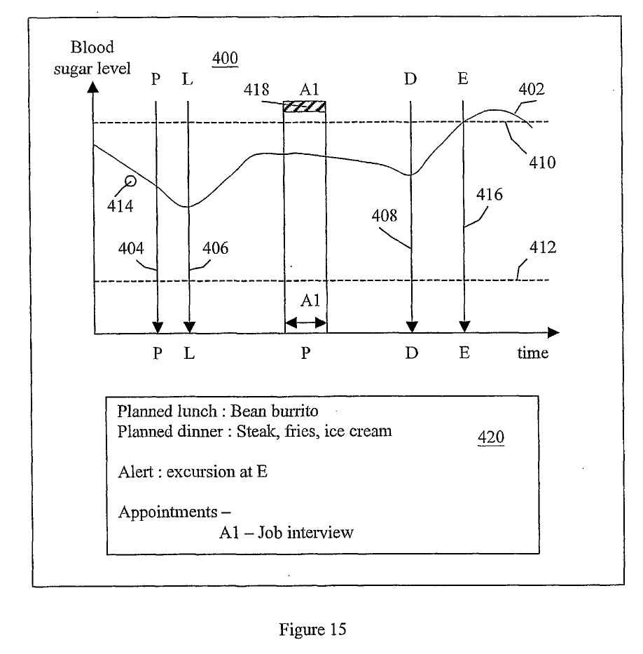

FIG. 15 shows a possible presentation on a screen or display of a computing device used to provide feedback to the person. Within the top half of the screen, a graph or chart 400 shows a curve 402, which represents the calculated blood glucose level for the person as a function of time. The current present) time P is indicated by arrow 404. Projected future levels of blood glucose are calculated using a glycemic response model, and are presented to the right of the arrow 404. The person has entered a planned lunch (L) and dinner (D), indicated at times shown using arrows 406 and 408 respectively. The nutritional content of these planned meals is used to calculate future values of blood glucose. As a result of the entered meals, the curve exceeds the upper recommended blood glucose level, shown by dotted line 410, after the planned dinner at a time E, shown by arrow 416. The previous accuracy of the model's predictions has been checked using a data point (414) which represents an actual blood glucose measurement for the person. As this point 414 is close to the curve based on previous food and blood glucose entries, the model is assumed to be accurate. If discrepancies are present, the curve can be shifted, the predictive algorithm modified, or the person recommended to perform more frequent blood glucose measurements. An appointment is indicated by region A1 at 418. Window 420 shows a summary of meals planned, and an alert regarding the blood glucose level excursion. Colors can be used to highlight events, such as appointments, the curve 402, and possibly also to highlight periods of time when the curve 402 goes outside the predetermined range bounded by upper limit 410 and lower limit 412.

Feedback is provided to the person on how to avoid this excursion above the upper acceptable limit. The person would also be warned about blood glucose level excursions below the lower limit 412, and advised on actions to take, such as increasing meal sizes, adjusting meal components, having snacks, changing the time and nature of exercises, and the like. The person would also be warned to avoid challenging activities during periods of low blood glucose.

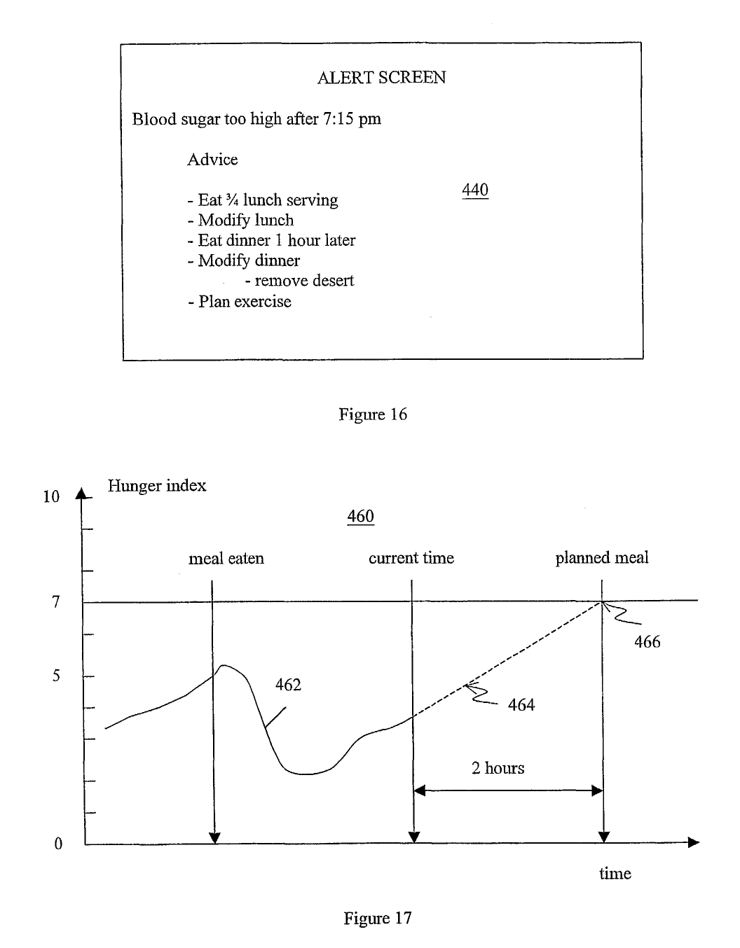

FIG. 16 shows a possible alert screen display 440, which may accompany the presentation of FIG. 15. Advice is given as text messages, though icons, graphics, and animations may also be used. Advice includes suggesting that the person not consume the full lunch serving, that lunch be modified, that dinner may be consumed at a later time, that dessert is removed from dinner, or that exercise is performed. These suggestions may be hyperlinked to other screens, allowing modifications to be entered. The effect of modifications on the blood glucose curve may be observed as modifications are entered. Other suggestions might include: optimum times for re-measuring blood glucose, and replacing high glycemic index meal components with low glycemic meal components. When selecting foods or meals from a menu, the software program may highlight foods and meals suitable for maintaining a healthy blood glucose range. For example, if a person knows that dinner will be at a late time, the time can be entered into the software program, and a combination of foods selected to maintain a healthy blood glucose level during the intervening time.

If the projected blood glucose curve has an excursion below a lower acceptable limit (or level), advice provided by the software program may include: suggesting a snack, suggesting the optimum time for a snack; canceling activities which may involve driving, significant decision making, or operating equipment; suggesting drinking water to maintain satiety (for example so as to maintain a weight control program); cancellation or rescheduling of fitness programs; changing meal times; increasing meal content; or adding low glycemic index foods to maintain a minimum blood glucose level over a protracted period. Glucagon injections may be suggested, or automatically administered by a device in communication with the PDA.

The software program may provide recommended upper and lower limits, for long term health goals, and essential upper and lower limits, which limit the range in which a person may be fully functional.

In one system embodiment, the glycemic response model is determined for a person using a glucose sensor and a PDA to record the time and content of meals. Once the glycemic response model has been determined, the glucose sensor may no longer be carried, and the system, in its simplest form, becomes a PDA loaded with a software program (such as diet log software) having a glycemic response modeling function. A person with diabetes would continue to use a blood glucose sensor, but it may not be necessary to test blood glucose levels as frequently as during the glycemic model determination, and so a different testing protocol may be used. If no continuous blood glucose monitoring is in progress, it might be advantageous if to use a physiological sensor to detect that a meal is being consumed, or has been consumed. The person may forget to enter meals into the PDA, and such a physiological sensor would enable the PDA to prompt for a reminder. Rising blood glucose levels may also be used to prompt a reminder to enter a meal.

Physical activity has been shown to be effective in reducing the severity of diabetic symptoms. However, in persons with diabetes like symptoms, physical activity can lead to dangerously low blood glucose levels (hypoglycemia). The person can be warned of such a possibility, either before or during exercise. Real time advice and feedback can be provided by the PDA. If blood glucose levels deviate from that predicted by models, then the person can be prompted by the PDA to enter any meals perhaps accidentally omitted. If the data entered is confirmed to be accurate, the model can then be modified by the actual response to the meal so as to improve the accuracy of the meal.

If glucose levels fall outside an acceptable range, as measured by a sensor in communication with the PDA, the person is warned by the PDA, for example by a vibration, alarm sound, flashing light, displayed message etc. If there is no response to the warning, the PDA may communicate with a remote device for example using a wireless internet connection to summon medical assistance.

Other physiological sensors may also be added to the system, in particular for hypoglycemic and hyperglycemic conditions. Physiological sensors may use colorimetry of the skin, skin conductivity, heart beat, electrical stimulation of extremities, or other parameters, individually or in combination, to determine the near-fainting condition associated with hypoglycemia, and the PDA may be used to provide advice to the person, or call for medical assistance. The PDA may also be used to control glucagon injections in such cases. Other physiological sensors, which may be advantageously added to systems described herein, are described elsewhere in this specification.

Calorie Balance

The software program can also be used to calculate the calorie balance for the person. Resting metabolic rate can be determined using an indirect calorimeter such as the gas exchange monitor (GEM) described by James R. Mault and others in pending U.S. application Ser. No. 09/630,398. If the calculated long term blood glucose indices are lower than measured, and also weight loss has been predicted by calorie balance data but weight has been gained, the problem is likely to be underreporting of food consumed. However, if the predicted weight change based on the calorie balance is accurate, but long term blood glucose levels are higher than predicted, there may be a problem with the glycemic response model (which can subsequently be modified) or there may be a medical problem with the person which can be diagnosed. There are significant medical and user accuracy advantages in combining a calorie balance calculation with a blood glucose level calculation. Weight change determination is more accurately performed in conjunction with bioimpedance measurement of hydration levels and body fat levels, as described in U.S. application No. 5,615,689.

The resting metabolic rate, and response of metabolic rate to activity, are important parameters in a weight loss program, and in the prediction of blood glucose and the hunger index discussed below. The metabolic rate may be determined using any of the following listed methods, or a combination of methods: using an indirect calorimeter; using equations based on physical parameters (for example height, weight), such as the Harris-Benedict equation; using improved equations based on physical parameters combined with demographic and/or body fat estimation; measuring physiological parameters which track metabolism, such as pulse rate, body temperature, and the like. Resting metabolic rate can be determined using an embodiment of the Gas Exchange Monitor (GEM) described in U.S. patent application Ser. No. 09/630,398, an indirect calorimeter invented by James R. Mault et al.

A person may also carry a physical activity monitor, adapted to provide a signal correlated with the physical activity of the person. This may be an accelerometer, GPS-based unit, physiological monitor (for example heart rate monitor, core temperature monitor, or respiration monitor), or the like, such as those described elsewhere in this specification.

A glycemic response model may include the effects of planned or performed activity, and resting metabolic rate. For example, the resting metabolic rate and activity-related energy expenditure can be determined and used to provide an estimated rate of reduction of blood glucose.

Closed Loop Glycemic Modeling

A software program having glycemic response modeling, diet log, and activity log functionalities can calculate average blood glucose levels over time, and hence calculate levels of glycohemoglobin and glycoproteins. These compounds are diagnostic of long term average blood glucose levels, and it is medically advisable that the concentrations of glycohemoglobin and glycoproteins are kept within predetermined healthy ranges. The calculated average blood glucose level determined using a software program can be compared with actual measurements of glycohemoglobin concentration or other components indicative of average blood glucose levels. Blood test may be carried out at intervals, for example once per month, per week, or at other intervals, which may be suggested by the software program or a medical professional. The average blood glucose level determined from the blood test can be compared with the calculated value from the glycemic response model provided by the software program. The glycemic response model can then be modified so as to provide more accurate blood glucose estimations.

A urine test is less painful than blood extraction, and may be more acceptable to the person. Urine can be analyzed for blood glucose using several techniques, for example as discussed in U.S. Pat. No. 6,166,807 to Kawamura et al. The measured levels of urine glucose can be compared with levels predicted by the software program using the calculated blood glucose curve. Urine glucose levels can be correlated with an average blood glucose level over the time before the sample was taken. Urine tests can be performed at intervals, such as once per day, or few days, or every week, and the average blood glucose values provided by the test compared with a corresponding average value provided by the software program. After any comparison of test results and software program predictions, the glycemic response model can then be modified so as to provide more accurate blood glucose estimations.

Embodiments of the present invention allow measurements of blood components (or other factors) correlated with an average blood glucose level over a period of time can be compared with the glycemic response model predictions for the corresponding average, allowing modification of the model so as to increase model accuracy. The comparisons between model predications and actual measurements can also alert the person to possible diet log recording problems. The software program can have algorithms to calculate instantaneous blood glucose levels over time (which can be compared with blood droplet tests), along with short term, mid-term, and long term blood glucose average levels.

According to U.S. Pat. No. 5,139,023, there is no correlation between glucose levels in saliva with blood levels. However, correlations may exist between saliva glucose levels and an average blood glucose level over a time period, in which case saliva glucose levels can be usefully monitored.

An advantage of glycemic response modeling is that average blood glucose levels can be calculated and compared with tests sensitive to long term average levels of blood glucose.

Blood glucose measurements can also be taken before and after eating a meal, for example by measuring blood glucose immediately before a meal and again a period of 0.5 to 2 hours later. The rise in blood glucose can be correlated with meal components, such as total carbohydrate content, form of carbohydrate, fiber level, and the like. A glycemic response model for the person can be developed comparing blood glucose levels made over time with foods recorded in a diet log. The glycemic response model can be continuously modified for improved accuracy by comparing model predictions with actual measurements.

Blood glucose measurements can be made by the person at any convenient time, and compared with the diet log records so as to provide a glycemic response model.

Insulin and Glucagon Administration

The software program can recommend insulin or glucagon injections, and can recommend an amount required and an optimal time for administration. The software program may be provided in different versions, for diabetics and non-diabetics, or the same version configured for use according to user requirements. Some persons will not use insulin, and so will not want insulin injections suggested. Others will use insulin, and therefore would want that option to be presented. The PDA can serve as an alarm to remind the person to administer medications. In a system embodiment, an automatic insulin or glucagon administration device can be used to provide the correct amounts of these compounds so as to maintain blood glucose within a predetermined range. For example, a microneedle array may be under wireless control by a PDA to administer as required by the person, based on the long term predictions of blood glucose using the glycemic modeling algorithm. Insulin may be added to interstitial fluid as it is returned to the body of the person after passing through an analysis module.

Hunger Index

An further aspect of this invention is the use of a hunger index in weight-control or physiological monitoring systems. The hunger index comprises a physiological component, for example blood glucose levels, blood hormone levels, satiety; and a psychological component. Satiety is related to feelings of fullness, related to stomach distention, and also to levels of certain hormones. For example, drinking a glass of water may make the person feel less hungry, even though it contains no calories. Certain appetite suppressants act by increasing satiety through for example swelling in the stomach, or affecting hormone levels. There may be some overlap between satiety and psychological components. The psychological component can include the time from the last meal, the time of day, and other terms correlated with factors which may be monitored using physiological sensors. The satiety component can include stomach distension, hydration levels, hormone levels, and other terms correlated with factors which may be monitored using physiological sensors.

The computerized nature of embodiments of the present invention may be used to reduce the psychological component of hunger index. For example, if a software program tells a person that they are not hungry, the person may be inclined to believe it. Such a psychologically manipulative element may be included into the hunger index calculating software. Obviously, the software program cannot misinform the person all the time, or it will lose credibility. But occasionally it might underestimate the hunger index, in order to help the person achieve a weight loss goal.

The hunger index can be a number, for example between 0 and 10. A value of 0 would mean the person is not hungry. A value of 10 might correspond to a extreme hunger, or a hazardous medical condition, sufficient for the PDA to request medical assistance for the person via access to a communications network. Intermediate values may correspond to a intermediate degrees of hunger. The software program running on a PDA (or other electronic device) uses the hunger index, combined with weight control goals (if any) to help the person plan meals and snacks. Negative numbers correspond to a gorged state, or excessive blood glucose levels in an non-diabetic person.

For example, the hunger index HI can be defined by a relationship of the form

HI=aG+bP+cS

where G is related to blood glucose levels, P is related to a psychological urge to eat, and S is related to satiety. The terms a, b, and c are chosen to combine the different effects on hunger in an appropriate manner, for example giving a value of HI that is, at most times, small (for example HI<10) and positive. The terms a, b, and c may be numerical constants, or numbers generated by equations chosen to model the effects of determined physiological parameters on hunger. The satiety and psychological terms can be combined into a single term. The hunger index will increase as satiety decreases, blood glucose levels decrease, and the psychological need to eat increases. The signs of the terms a, b, and c in Equation 1 are chosen to reflect this behavior.

The physiological component of the hunger index can be measured through physiological monitoring, such as blood glucose monitoring, blood hormone monitoring, stomach distention monitoring, etc., or predicted by combining diet logging with activity level monitoring and the knowledge of the person's resting metabolic rate. The person's caloric need may be calculated from their metabolic rate and physical activity level. Their caloric intake can be calculated from diet logging. Using the calculated caloric balance (deficit or surplus), combined with some individualized modeling, the hunger index may be calculated. The individualized modeling may include the person's response to food, such as digestion rate, blood glucose response, time of day, etc. An accurate determination of resting metabolic rate is very important for an accurate calorie balance determination. The Gas Exchange Monitor (GEM), an indirect calorimeter invented by James R. Mault, is an important step in resting metabolism determination without the need for expensive and/or complicated equipment.

The hunger index estimated by the PDA may be compared with the hunger index the person thinks they feel. The model for the person may then be refined, particularly the psychological component. Perhaps a person tends to feel hungrier in the evening, for no good physiological reason. If a person is hungry, but the caloric balance is in surplus, and there is no danger of dangerously low levels of blood glucose, the PDA may instead suggest alternatives to highly caloric foods, such as an activity, other distraction, calorie-free gum, etc. The PDA may initiate an insulin injection for a person with low blood glucose utilization due to insulin shortages.

Food response indexes may also be assigned to food, drinks, and medications, according to their effect on the hunger index of the person. For example, the food response index (FRI) will be related to its effect (i.e. increased level) on blood glucose levels. Bulky foods give feelings of satiety. Also, foods which are consumed slowly may have beneficial psychological benefits, as well as increasing satiety. These effects are time dependent, so in computer models of the person, the hunger index of the person, and the food response index of food, are more accurately described by time-dependent variables, related to total available blood glucose, glycemic index, and the decay rate of blood glucose after it has reached a maximum. Appetite suppressants may also be included in the model, as they may reduce feelings of hunger through stomach-filling effects, or effects on hormone levels, the brain, etc. Hence, a method of assigning a food response index to a food, for a person eating the food, the person having a physiology and a psychology, comprises: determining the effect of the food on the physiology of the person; determining the effect of the food on the psychology of the person; and combining the physiology effect and psychology effect into a food response index, whereby the food response index of the food is used to predict the effect of the food on the hunger of the person.

The response of the person to certain foods can be monitored in detail, for example using sensors, questioning, blood glucose monitoring, and other physiological monitoring, and used to provide a model of the person's hunger index response to eating those foods.

Hence, a hunger index can be assigned to the person by determining a blood glucose level, a satiety level, and a psychological level for the “need to eat” for the person, and combining the determined levels using an empirical relationship such as given above in Equation 1. The levels may be determined by actual measurement using sensors, testing, etc., using a model for the person, estimation, etc.

Similarly, a food response index may be assigned to a food, perhaps individualized to a specific person eating the food, by determining the effect of the food on the physiology of the person, the psychology of the person, and by combining the physiologic and psychological effects. A bulky food will have a higher food response index than a non-bulky food with the same caloric content, due to the effect on the satiety of the person.

An underlying glycemic response model can be developed for the person and used in the prediction of hunger index. The person's blood glucose and insulin response to meals can be measured for certain meals, then captured in an individualized model for that person. The glycemic response model, in conjunction with a diet log, allows calculation of a long term average blood glucose level. The calculated level can be compared with glycohemoglobin measurements, and the comparison used to refine the model. Environmental conditions, such as temperature, may be included in the hunger index model for a person.

The PDA system also has the capability of predicting future hunger levels of the person over time. Meals consumed, and meals planned, may be known to the PDA. Using a previously determined physiological response model of the person, future blood glucose levels of the person may be predicted. If blood glucose levels are predicted to fall to low levels, corresponding to high hunger index, a pre-emptive snack may be suggested.

FIG. 17 shows a graph 460 showing how the hunger index may vary with time for a person. After a meal is consumed, the hunger index falls as shown by curve 462, and then starts to rise again. At a time 2 hours before the next planned meal (an arbitrarily chosen time period for illustrative purposes), the software program running on the PDA is used to plan the meal. Projecting the person's hunger index to the planned meal time, as shown by the dashed line 464, the software program running on the PDA recommends a meal with a total food response index of 7. The line 464 can be generated by simple extrapolation, or using a hunger index model for the person, for example including a glycemic response model as described elsewhere.

The PDA can also be used to predict the fall in hunger index after a meal is consumed. For example, after a meal which increases blood glucose levels relatively slowly after eating (as quantified by its glycemic index), for example one with many complex carbohydrates, the PDA may indicate something along the lines of “Your hunger index in 6 now, but wait 20 minutes and it will fall to 3”. The feedback provided by the PDA may be in many forms, visual, audio, etc.

For example, the PDA suggests meals or snacks based on the hunger index of the person. Food response indexes may be assigned to foods in a database, and matched with the hunger index of the person. For example, if the person's hunger index is low for example 1 out of 10 (1/10), the PDA may suggest a celery stick as a snack. If the hunger index is higher for example 8/10, the PDA may suggest moving dinner earlier, and planning a snack for some time after dinner to prevent high hunger indexes from occurring again.

The PDA may assist ordering food based on the hunger index. The food may be for delivery, pick up, or for eating at a restaurant. For example, it may order a low-fat stir-fry from a local restaurant with delivery, via the Internet, and charge the cost of the meal to an account. If the PDA has phone capabilities, it might suggest items and phone numbers from local restaurants. If supplies of often-consumed foods are running low, the PDA may order them to be delivered from a local grocery store. Prepackaged foods may be ordered from businesses specializing in weight control. The PDA may provide menus and directions to local restaurants, using a combination of Internet database access and GPS technology.

The PDA may also suggest drugs or medications to subdue hunger levels. For example, it might suggest a cup of coffee and a cookie. The caffeine in the coffee may help reduce the hunger index of the person. Appetite suppressants may also be suggested. The time-dependent effect of appetite suppressants may be included in a physiological model of the person used to predict glycemic response, weight loss, and hunger index.

The PDA can suggest optimum times for activities, or can suggest stopping an activity. For example, exercise can be suggested when it would not decrease blood glucose levels to a point where the hunger index would be distracting. Demanding activities, such as operating machinery, should cease if the hunger index is too high.

The PDA may also be in communication with a remote computer system, for example the Internet. Advice from a computer expert system, physician, web site, etc. may be displayed to the person as text, graphics, video, etc., on the PDA or other display device such as an interactive television.

Glycemic Response Algorithm

The body's response to carbohydrate ingestion is complicated. A summary of processes and problems is described in U.S. Pat. No. 6,122,536 to Sun et al., herein incorporated by reference. The glycemic response model used in a software program may only approximate the actual body response to food ingestion.

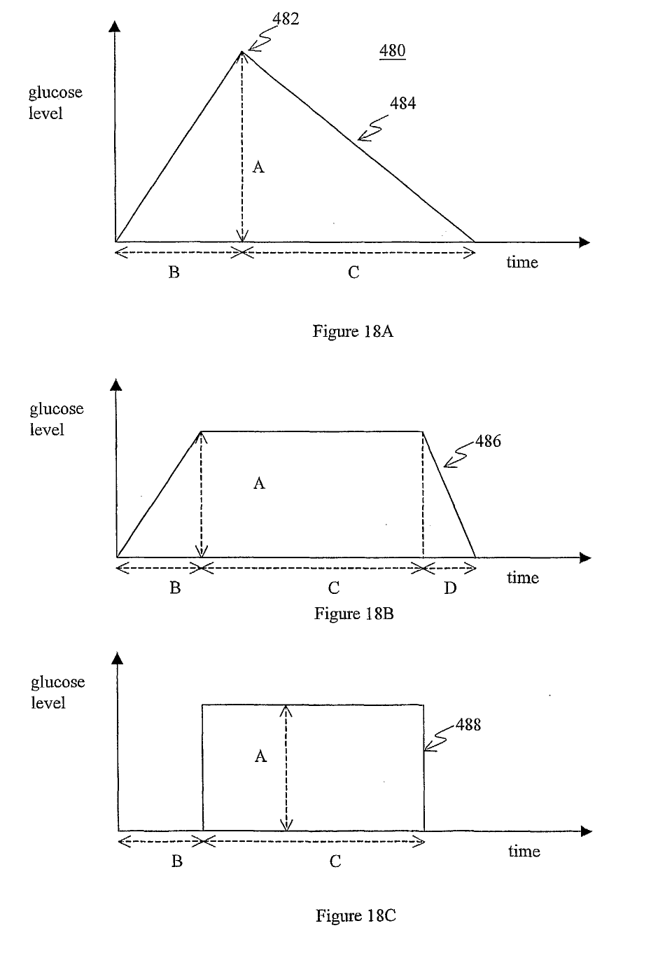

FIG. 18A shows a graph 480 showing a model blood glucose response curve 482 (equivalently, a model glycemic response curve) against time after a meal is consumed. This simplified curve has a peak level 482 above the initial level, characterized by a glycemic response parameter A, which is related to the increase in blood glucose above a baseline level, the baseline here assumed not to change. The term A is correlated with the conventionally understood definition of glycemic response, namely the rise in blood glucose for a particular food, usually expressed as a ratio to a standard such as white bread or glucose. The glycemic response parameters A, correlated with a plurality of foods, and used in a glycemic response model for the person, can be modified by a glucose tolerance test performed on the person. For example, an individual scaling parameter for the person, determined by the glucose tolerance test, can be applied to conventional glycemic index values. Conventional methods may not provide accurate future predictions of blood glucose. The glycemic response parameter B is related to the rise time to peak level, and the glycemic response parameter C is related to the decay time of blood glucose back to the baseline. The area under the curve, in this case a triangle, is 0.5(A×(B+C)), and this area is correlated with the total available glucose from the food, and hence is correlated with portion size. By making an assumption about the curve (such as assuming C=2B), a glycemic response curve can be generated from a portion size and a single glycemic response parameter (per unit quantity of food) related to the value of A. For small portions, the area under the curve can be assumed to be directly proportional to portion size, and the parameter A can be scaled linearly with portion size. For larger portions, the induced insulin from consuming the food may reduce the area under the curve by some factor, so that the area under the curve has a non-linear relationship with portion size.

Blood glucose is removed from the blood stream at times of excess, and added at times of need. These physiological responses can be modeled by making assumptions such as that the rate of blood glucose removal is proportional to the concentration excess above a predetermined level. Insulin production can also be assumed to follow a similar behavior. Glucagon release can be assumed to have a rate of production related to the concentration deficit below a predetermined level. Physical activity will increase the rate of removal, and this effect can be included in a model. Values of A (and possibly B and C) can be stored in a database, and associated with components of a meal or food. The total response can be estimated by summing components according to presence. Some foods, such as fiber, may modify the parameters of foods consumed concurrently, which can be included in the model.

FIG. 18B shows another model blood glucose response curve (486), a trapezoid having parameters A (a plateau height, related to blood glucose increase), B (a rise time), C (a plateau time), and D (a fall time). The number of independent parameters can be reduced by making assumptions about the interdependence of B, C, and D, for example assuming B=D=0.3 C, or some other relationship between two or more of B, C, and D. FIG. 18C shows a further model blood glucose response curve (488), having a plateau height A, a delay time B, and a plateau time C. Other model forms can be used, such as semicircles, half-ellipses, curves, other geometric forms, analytical curves with variable parameters, numerical fits to experimental data, and the like.

After determining model blood glucose response curves to a plurality of foods, the blood glucose response parameters for the foods can be stored in a memory of the PDA, a memory module to be inserted into a PDA, or at a remote memory location accessible through a communications network. Blood glucose response parameters for various foods can be initially generated for a person based on demographic factors, such as age, gender, weight, and height, then modified by comparison of a person's actual blood glucose levels with the predicted levels based on modeling using the parameters.. A software program having a glycemic response modeling function can use the blood glucose response curves known for plurality of foods to calculate a blood glucose response from the meals recorded in a diet log.

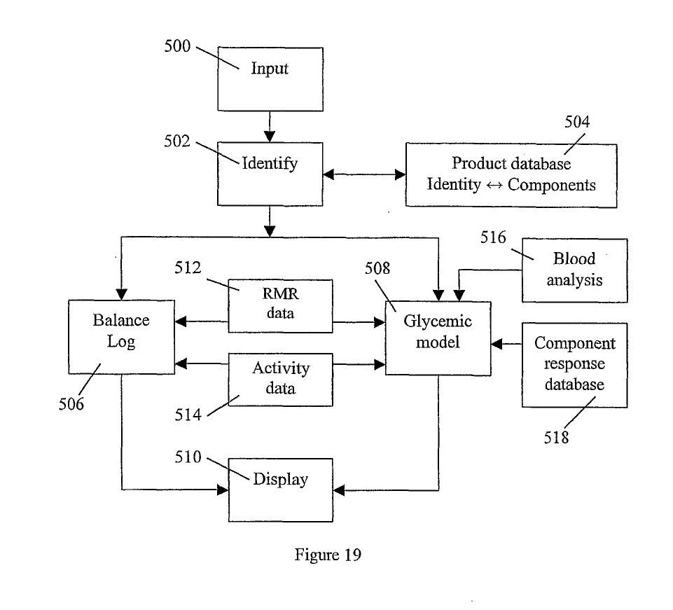

FIG. 19 shows a flowchart representing a software program running on a computing device. Box 500 represents the person entering food consumed, using any appropriate method. Box 502 represents the software program providing a food identification, for example by correlating an entered code with food identification and data. Box 504 corresponds to food identification being provided to a database 504, with corresponding nutritional data being extracted from the database. Box 506 corresponds to some part of the nutritional data, for example caloric data, being passed to a balance log calculator which calculates the calorie balance of the person from food consumed, activity levels, and metabolic rate. Box 512 corresponds to providing RMR data, for example as determined using an indirect calorimeter. Box 514 corresponds to providing activity data, for example from an activity monitor. Box 510 corresponds to displaying calorie balance and projected blood glucose levels on display 510. Box 508 corresponds to determining blood glucose levels for the person, using nutritional data, activity levels, metabolic rate, blood analysis data, and blood glucose response parameters corresponding to nutritional data provided by a component response database. Box 518 corresponds to providing blood glucose response parameters from a database, corresponding to nutritional data received. Box 516 corresponds to performing a blood test at intervals, and providing the data to a glycemic response algorithm.

A single database can be used, in which food components are correlated with food identity, and at least one glycemic response parameter. The database can contain glycemic response curves parameterized for various foods, which may be established using a glucose tolerance test, and refined using a closed loop system in which measurements refine the prediction based on the database contents. Different response curves may be determined for simple sugars, complex carbohydrates, fiber-rich foods, food or otherwise treated to reduce the glycemic response peak, and other food types.

Physiological Monitoring Systems

As part of a physiological monitoring system, a portable computing device can be used to monitor a number of factors influencing the physiological state of the person. These factors are discussed below. Some or all of the following categories may be logged. Diet and activity are the most important. Remote devices and sensors can be in communication with the PDA, for example using Bluetooth wireless communication protocol (Ericsson AB). Electrical, electromagnetic, and manual data entry may also be used, along with the transfer of non-volatile memory media, voice recognition software, etc. The person may enter data manually into the PDA. Optical character recognition may be used for entry of numbers and text into the PDA. Certain sensors may be incorporated directly into the PDA.

Diet Log

Diet logging can be achieved using any convenient method, such as one of the following methods, or some combination (for convenience, the logging device is referred to as a PDA, but may be any portable electronic device, as discussed above).

Food consumed can be entered into the PDA using a menu entry system, for example using key presses, stylus entry, voice entry, eye motion detection, hand motion (gesticulation) detection, etc.—for voice entry, a restricted voice recognition capability may be adopted for computational simplicity, for example one which recognizes only numbers or letters associated with menu choices, such as disclosed in co-pending provisional application serial No. 60/212,319 filed Jun. 16, 2000, to James R. Mault. The name, code number, other identifier, time of consumption, nutritional data, portion size, or other information concerning food consumed can be entered into the PDA, for example using key presses; stylus entry such as handwriting recognition systems); voice entry; optical character recognition (OCR), for example by placing an item such as a food package, receipt for a food item, a menu, and the like near an OCR-adapted PDA (the PDA may have in-built imaging and OCR capabilities, or be in communication with an accessory device, such as a pen-like device which can be scanned over text, or an imaging device used to image and recognize text); bar-code scanning of an item (for example a food package, receipt, menu, etc.) to obtain identification of food consumed and hence nutrition information, or imaging of a bar code for similar purposes; receiving data transmission from the package, such as from a wireless transponder embedded in a food package under interrogation from the PDA; reading of data from magnetic strips on a food package, menu, receipt, etc.; reading data from patterns on the surface of the food or package, for example microstructures, holograms, etc.; entering data from menu listings, for example using key press entry, stylus entry, optical character recognition, barcode entry, etc. including portion size, food identification, or full or partial nutrition information; receiving data transmissions for example wireless, IR, optical, memory storage media, etc., from food vendors (for example grocery stores, restaurants, etc.), with the data stored for future use if the food is not to be consumed at the time of purchase; receiving data over a communications network, for example the Internet, for example receiving nutrition information on items purchased; recording voice records of food consumed.

Food can be imaged using a digital camera, for example using a PDA adapted to obtain images, or a PDA in communication with an imaging device. Images may be stored for later analysis by the person, computer expert system, or other person, or be transmitted via a computer network for remote analysis by a person or computer. An image recognition system on the PDA, or on another device in communication with the PDA, may also be used. For example, the person might image all foods consumed. The images may then be transmitted via a computer network to a remote computer system where the foods consumed are identified and stored in a database, where the person may access them. Optical character recognition may also be used to obtain product identification and nutritional information from package labeling.

Further, foods and food components can be identified by other techniques, such as: spectroscopy or spectroscopic imaging (for example imaging over IR-visible-UV spectral ranges), which may also assist image recognition software to identify foods (foods may contain spectroscopic markers to aid identification); using electronic nose technology, using for example olfactory sensors in the PDA; using the physiological response to food, for example by monitoring the change in blood glucose, triglycerides, or other blood components, or using other physiological responses to eating; and combustion or vaporization (for example laser vaporization) of food samples, followed by vapor, condensate, or residue analysis; calorimetry of food for example using a microchamber.

The portion size of food consumed may be estimated by one or more the following methods, possibly in combination with method(s) listed above. The following methods may be used, individually or in combination: weighing the food item using scales built into or in communication with the PDA (an attachment for the PDA may be adapted for supporting or hanging a food item, and for obtaining the weight of the food item); estimating the weight of the food item, either by the person, by another person such as a food vendor, or from previously measured data for similar food items; prodding the food item, so as to estimate the mass of the food item, for example using a mechanical prodding accessory to the PDA (different prodding forces may be used to distinguish inertial response from frictional effects); measuring the inertial effect of holding the food item on the motion of the hand or wrist of the person, and hence the mass of the food item, using accelerometers mounted on the person; determining the attenuation of radiation, for example electromagnetic radiation, ultrasound, etc., by the food item, so as to estimate the density and/or size of the food item; reading the weight off the package and manually entering it into the PDA; determining the weight using a product identifier, such as a bar-code, numerical code, product name, etc; determining weight using a scale, and manually entering the data into the PDA. Further, the size of a food item can be estimated using an imaging method. The focusing action of the imaging device may be used to estimate the image scale. The person may use a standard fork, or other object, to provide image scale. Imaging may be from more than one angle, followed by dimensional analysis. Images may be transmitted to a remote location over a communications network in order for some other person or computer system to estimate food nature or portion size. The terms food, food item, portion, or food portions may refer to a food package, food serving, drink serving, and the like.

Activity Log

The PDA can be used for monitoring the physical activity of the person. The PDA is used to receive and store activity-related data, for example, the PDA may be used for: receiving signals from a body mounted accelerometer, a pedometer, a posture sensor, or a similar device (or devices) which provides a signal (or signals) related to the activity level of the person; receiving signals from an array of body mounted accelerometers; receiving data from an exercise machine, during or after exercise by the person, relating to the energy expended by the person; receiving data from a personal trainer (either human, robot, or computer) related to energy expended in an exercise program; receiving positional and/or altitude data, using a global positioning system (GPS), triangulation method, or other positioning scheme, to obtain data such as speed, distance traveled, distance climbed, and hence energy expended in walking, running, cycling, swimming, etc; receiving data from an altimeter to estimate the effect of altitude on metabolism; receiving data from physiological monitors which obtain information related to physical activity, such as pulse rate, respiration frequency, body temperature, respiration volume, respiration frequency, etc; estimating physical activity of the person from the responses of the person to questions, for example quizzes given by the PDA; receiving data from an indirect calorimeter to obtain metabolic rate in the resting state, and possibly also in the active state, for estimation or direct determination of total energy expenditure; receiving data from any sensor providing a signal related to physical activity which has been calibrated using an indirect calorimeter; receiving data from muscle contraction sensors; and receiving data from posture sensors related to physical activity, such as from skin-mounted ultrasonic transducers.

Data may be received by the PDA using any convenient method, for example wireless communication (for example Bluetooth), IR beams, wires, cables, electrical interfaces, data storage media transfer, voice entry, manual entry (for example using keys or stylus), etc. Sensors may be part of the PDA, in which case signals would be received by connections contained within the housing of the PDA. Data may be received in continuously, in real time, or at periodic intervals. The PDA may prompt a sensor to send data.

Physiological Monitoring

A PDA can also be used to monitor the physiological state of the person. It may communicate or otherwise receive data from physiological sensors or monitors carried by the person. It may also receive data from other physiological sensors or medical diagnostic equipment, for example devices located at a gym, doctors office, shopping mall, retail space, etc. The following methods or devices may be used: monitoring body fat proportion, for example using bioimpedance; monitoring skin resistance; monitoring blood component levels, for example glucose, oxygenation levels, carbonyl groups, ethanol, etc.; monitoring insulin, glucagon, or other hunger-related blood component levels, or other body products related to weight and metabolism, for example lactate, glycogen levels, epinephrine, norepinephrine, and thyroid hormone; monitoring levels of compounds related to long-term average blood glucose levels, for example glycohemoglobin levels in the blood; monitoring blood pressure; monitoring blood pH, carbonate ions, and ketone bodies in the blood (ketone body levels are related to fat metabolism, and monitoring is useful in a diet or exercise program); monitoring leptin levels, relating to satiety and obesity in humans; monitoring levels of uncoupling proteins (UCP1, UCP2), UCP regulating compounds, and analogs, which relate to metabolism; monitoring gastrointestinal hormones and hormone regulators, (for example cholecystokinin, gastrin, etc.), and antibody/antagonist levels, which relate to digestion efficiency; monitoring the blood for disease-related components, for example liver enzymes, which relate to liver disease; monitoring the blood for products of protein metabolism, such as nitrogen-containing compounds for example urea (as protein metabolism is generally unwelcome in for example weight control programs as it leads to muscle loss and a decrease in resting metabolic rate); monitoring the blood for pathogens for example bacteria, viruses, protazoans, and/or antibodies to them; monitoring the blood for parasites, for example merozoites (malaria); monitoring the blood for viruses (and/or antibodies) related to obesity, for example adenoviruses such as Ad-36; monitoring vitamin, mineral, and electrolyte levels; monitoring pulse rate; monitoring respiratory volume, for example using abdominal expansion, acoustic monitoring, or a spirometer; monitoring for respiration abnormalities, for an awake or asleep person, for example monitoring sleep apnea; monitoring exhaled air for evidence of lung infection or disease; monitoring hydrogen levels in exhaled air, which relates to digestion efficiency; performing respiratory analysis, for example for oxygen consumption, or exhalation of carbon dioxide, ketones, aldehydes, nitric oxide, ammonia, other nitrogen-containing compounds, peroxides, sulfur-containing compounds, esters, or other volatile organic compounds, which may be symptomatic of diabetes, liver misfunction, kidney misfunction, etc.; respiratory monitoring of acetone to indicate the onset of fat burning in a diet and/or exercise program, or to indicate diabetic symptoms; respiratory monitoring of nitric oxide (NO) as indicator of inflammatory airway diseases (for example asthma), for example as described in U.S. Pat. No. 6,010,459; respiratory monitoring of isotopically labeled gases, for example due to the metabolism of labeled compounds, for example as in U.S. Pat. No. 5,962,335; monitoring brain activity (brain waves), and using the physiological effects of thinking to control devices (for example U.S. Pat. No. 6,024,700); monitoring the heart, using for example using electrocardiograms, acoustic monitoring, etc., also forming correlations between data sets, filtering, signal processing, etc.; monitoring body temperature; monitoring skin temperature, this may decrease at the extremities in some physiological conditions; monitoring physiological indications of eating, for example blood glucose, body temperature, swallowing, etc. which may assist diet logging; monitoring posture, for example using ultrasonic transducers which determine distances between each other, which can also be used to monitor physical activity; monitoring time awake, or time asleep; monitoring time engaged in a specific activity (occupational or recreational), for example driving, operating equipment, walking, running, other exercise, typing, etc., using one or more physiological states; measuring body weight, for example using scales in communication with the PDA, or entering data into the PDA; monitoring skin pallor (for surface blood flow estimation, this reduces in hypoglycemic state); receiving data from other analytical or testing devices; monitoring response to electrostimulation, particularly at the extremities (this reduces in hypoglycemia); recording emotion (for example heartbeat, facial muscle state, stress hormone level); monitoring hormone levels in the blood; monitoring perspiration level; performing perspiration analysis; transmitting physiologic data to a physician, nutritionist, dietician, fitness consultant, or other professional, using for example a wireless Internet connection, for example transmitting data at periodic intervals or on demand from the PDA to a database on a remote (i.e. not carried by the person) computer which may be accessed by authorized persons; performing urine analysis (for example for quantifying nitrogen metabolism, estimating protein intake, etc.); monitoring metabolic rate, for example using an indirect calorimeter; monitoring bone density, for example using ultrasonic transducers mounted on the skin using an adhesive clip and/or acoustic impedance matching gel (the person may be prompted to take calcium supplements if bone density falls below a certain level); searching for tumor markers; detecting gene sequences; and monitoring pupil dilation. Further, hydration level can be monitored. For example, the current Handspring Visor, or similar device, may be provided with a frame plugging into the Springboard slot, with four electrodes projecting from the frame, and appropriate electronic circuitry provided for bioimpedance analysis of the person's body. Bioimpedance data may be used to determine body fat content, and can also be used to monitor short-term hydration level changes, over time periods during which body fat proportion would not be expected to change. Dehydration is known to sometimes stimulate the appetite.

Sensors for Physiological Monitoring System

Monitoring may be carried out using physiological sensors placed on the body of the person, implanted or embedded into the body, swallowed, or carried as portable devices. Physiological sensors may be powered by batteries, solar power, radio waves emitted by another device, low frequency (in the US, typically 60 Hz) electromagnetic radiation due to power transmission, wireless station output (for example using transmissions from one or more high-power AM radio stations to provide, after reception and rectification, a d.c. voltage so as to power a transducer or sensor), thermoelectric effects (for example based on temperature gradients near the skin surface), or other means. Data may be transferred from sensors to the PDA using manual data entry into the PDA, IR links, wireless links (for example Bluetooth protocol), cables, voice entry, nonvolatile memory media (for example flash cards), etc. In some cases, tests may be carried out by the person or a health worker, and the results entered into the PDA, for example using a menu-based entry system. Physiological and other data obtained using other systems may be transferred to the PDA using an Internet connection, wireless communications, wires, IR, interfaces, etc. Data may also be transferred to a communications system, web-site, database, etc. with which the PDA communicates.

As part of a physiological monitoring system, a person may use one or more of the following methods: wearing a wristwatch-like device containing sensors; embedding or implanting sensors in the body; wearing patches on the skin; detecting color changes of elements in contact with the body; extracting and analyzing blood, for example using an automated device; wearing belt-mounted devices; wearing sensors mounted on a band circling some part of the body, wearing an earring or other body-piercing element adapted so as to monitor for example the blood; carrying a skin-mounted or belt-mounted sensor with the form and electrical connections of a non-volatile memory card (or “flash card”), such as manufactured by Sony, SanDisk, and others, which may also monitor physical activity; carrying microphones for example so as to monitor cardio-vascular noises; having electrodes contacting the skin, for example for bioimpedance analysis; and drawing blood and carrying out various analytical techniques, for example using enzymes.

Medication Log

A PDA may be used to monitor medication intake, or to help control the administration of medication. The following methods may be used, either individually or in some combination: entering data manually into the PDA, for example concerning medications taken; reading barcodes on medicine containers, or on the tablets themselves; imaging tablets, for example if tablets are spectroscopically (for example optically) or shapewise distinct; receiving data from injection or administration devices, perhaps controlled by the PDA, for example remote controlled insulin pumps, syringes, tablet dispensers, etc.; providing the person with feedback or reminders, for example instructions to take medicine, using software on the PDA; monitoring the administration of appetite suppressants to the person, monitoring of medication levels in the person's body, possibly coupled with links to dispensing devices (for example controlled syringe administration of drugs such as insulin), or to feedback to the person to take medication; warning the person of side effects of medications, for example using a database accessed by the PDA using a wireless internet connection; warning of dangerous drug interactions, if multiple medications are being used, for example using a remote database; transmitting medical/dietary/physiological data to a physician; receiving data over a communications network, for example the Internet, based on prescriptions or purchases; and transmitting medical/dietary/physiological data to a remote database, where it may be accessed by any authorized person.

Environmental Log

The PDA may be used to monitor environmental conditions, such as those in the following list. Appropriate sensors may be part of the PDA, carried with the person by some other method, or otherwise be appropriately located for transmission of useful information to the PDA. The following methods may be useful, either individually or in combination: recording ambient temperature; monitoring radiation levels; monitoring smoke or particulate levels; monitoring atmospheric pollution levels for example ozone, nitrogen oxides, chemical solvents; monitoring noise levels; recording body cooling rates, wind-chill, etc. for example from skin temperature; monitoring light levels; monitoring UV levels; determining location (from GPS or other positioning method); cosmic ray levels (for example for aviation employees); monitoring odor levels; monitoring water pollution; receiving data over a communications network such as the Internet concerning environmental conditions (for example air pollution, pollen count, etc.); manual entry of data from diverse sources, for example readings from sensors, data from publications (newspapers, web-sites, etc.); and monitoring altitude.

Other embodiments of the invention will be clear to those skilled in the art. Having described our invention, we claim:

1. A system for assisting a person to maintain a blood glucose level between predetermined limits, comprising:
electronic apparatus including:
data entry means enabling entering into the apparatus nutritional data of foods consumed, or proposed to be consumed, by the person, and the time consumed or proposed to be consumed;
a display;
a clock;
a memory for storing a glycemic response model representing time-varying changes in glycemic response for the person based on representative foods having representative nutritional contents;
a data processor;
and a software program executable by said data processor:
to process the nutritional data and times thereof entered via said data entry means with respect to said stored glycemic response model;
to generate a time-varying glycemic response curve based on the entered nutritional data;
and to display said generated glycemic response curve with an indication of said predetermined range of blood glucose level, and thereby to alert the person with respect to any deviation of the generated glycemic response curve from said predetermined range.

2. The system according to claim 1, wherein said software program executable by said data processor also displays alert messages alerting the person to specific deviations in the generated glycemic response curve from said predetermined range.

3. The system according to claim 1, wherein said software program executable by said data processor also displays advice messages advising the person as to recommended changes in the foods to be consumed by the person, or the times thereof, in order to maintain the blood glucose level within said predetermined range.

4. The system according to claim 1, wherein said memory also stores therein the nutritional values of a number of food items; and wherein said software program executable by said data processor also selects the nutritional values of the food items entered into the data processor via said data entry means for use thereof in generating said displayed time-varying glycemic response curve.

5. The system according to claim 1, wherein:
said stored glycemic response model is also based on representative physical activities of the person;
said data entry means also enables entering physical activity data regarding physical activities performed, or proposed to be performed, by the person, and the times thereof;
and said entered physical activity data is also used for generating said time-varying glycemic response curve which is displayed in said display.

6. The system according to claim 5, wherein said memory also stores therein physical activity values of a number of physical activities by the person; and wherein said software program executable by said data processor also selects the physical activity value of the physical activities entered into the data processor via said data entry means for use thereof in generating said time-varying glycemic response curve.

7. The system according to claim 1, wherein said software program executable by said data processor includes a glycemic response algorithm which predicts the effects of representative food items on a person's blood glucose level in order to generate said glycemic response model stored in said memory.

8. The system according to claim 1, wherein said software program executable by said data processor determines said glycemic response model stored in said memory by utilizing actual measurements of the blood glucose level of the person before a food item of known nutritional content is consumed by the person, and at a plurality of spaced time intervals after said food item is consumed.

9. The system according to claim 1, wherein said software program executable by said data processor determines said glycemic response model stored in said memory by maintaining a log of meals consumed by the person over a period of time, and utilizing actual measurements of the blood glucose level of the person at a plurality of spaced time intervals after means are consumed by the person.

10. The system according to claim 1, wherein said software program executable by said data processor also utilizes demographic factors associated with said person for determining said glycemic response model stored in said memory.

11. The system according to claim 1, wherein said software program executable by said data processor determines said glycemic response model stored in said memory by utilizing actual measurements of the blood glucose level of the person after consuming food items having known nutritional contents.

12. The system according to claim 1, wherein the person is a diabetic receiving an insulin control drug, and wherein said software program executable by said data processor determines said glycemic response model stored in said memory by utilizing actual measurements of the blood glucose level of the person after receiving a known dosage of the insulin control drug.

13. The system according to claim 1, wherein said electronic apparatus is embodied in a portable computer unit carried by the person.

14. The system according to claim 1, wherein said data entry means and said display are embodied in a portable computer unit carried by the person, and said data processor, clock and memory are embodied in a data processor at a remote location communicating with and serving a plurality of said portable computer units.

15. A method of assisting a person to maintain a blood glucose level within a predetermined desired range, comprising:
storing in a data processor a glycemic response model representing, time-varying changes in glycemic response for the person based on representative food items having representative nutritional contents;
entering into the data processor nutritional data of foods consumed, or proposed to be consumed, by the person, and the times thereof;
processing said nutritional data and times thereof with respect to said stored glycemic response model to generate a time-varying glycemic response curve based on the entered nutritional data;
and displaying said generated glycemic response curve with an indication of said predetermined range of blood glucose level to alert the person with respect to any deviations in the generated glycemic response curve from said predetermined range.

16. The method according to claim 15, wherein said data processor also displays alert messages alerting the person to any specific deviations in the generated glycemic response curve from said predetermined range.

17. The method according to claim 15, wherein said data processor also displays advice messages advising the person as to recommended changes in foods to be consumed by the person, or the times thereof, in order to maintain the blood glucose level within said predetermined range.

18. The method according to claim 15, wherein said data processor also stores therein nutritional values of a number of food items, and selects the nutritional values of the food items entered into the data processor for generating said displayed time-varying glycemic response curve.

19. The method according to claim 15, wherein:
the glycemic response of said stored glycemic response model is also based on representative physical activities;
said data entry means also enables entry into the data processor physical activity data of physical activities performed, or proposed to be performed, by the person, and the times thereof;
and said generated and displayed time-varying glycemic response curve is also based on said entered physical activity data.

20. The method according to claim 19, wherein said data processor also stores therein physical activity data for a number of physical activities by the person, and selects a specific physical activity data according to specific physical activities performed, or proposed to be performed, in the data processor, as entered via said data entry means, for generating said displayed time-varying glycemic response curve.

21. The method according to claim 15, wherein said glycemic response model stored in said data processor is determined by maintaining a record of foods consumed by the person, and utilizing a glycemic response algorithm which predicts the effects of consumed food items on the person's blood glucose level.

22. The method according to claim 15, wherein said glycemic response model stored in said data processor is determined by measuring the actual blood glucose level of the person before a food item of known nutritional content is consumed, and at a plurality of time intervals after said food item is consumed.

23. The method according to claim 15, wherein said glycemic response model stored in said data processor is determined by maintaining a log of means consumed by the person over a period of time, and measuring the actual blood glucose level of the person at a plurality of intervals after means are consumed.

24. The method according to claim 15, wherein demographic factors associated with said person are also utilized for determining said glycemic response model stored in said data processor.

25. The method according to claim 15, wherein said glycemic response model stored in said data processor is determined by measuring the actual glycemic response of the person after consuming food items having known nutritional contents.

26. The method according to claim 15, wherein the person is a diabetic, and said glycemic response model stored in said data processor is determined by measuring the actual glycemic response of the person after receiving a known dosage of an insulin control drug.

27. The method according to claim 15, wherein said nutritional data is entered, said data is processed, and said generated glycemic response curve is displayed, in a portable computer unit carried by the person.

28. The method according to claim 15, wherein said nutritional data is entered and said generated glycemic response curve is displayed, in a portable computer unit carried by the person, and wherein said data is processed in a remotely-located data processor which communicates with and serves a plurality of said portable computer units.
